(19) United States
(12) Patent Application Publication
(10) Pub. No.: US 2025/0222024 A1
(43) Pub. Date: Jul. 10, 2025

(54) METHOD AND COMPOSITION FOR TREATING CORONA VIRUS, INFLUENZA, AND ACUTE RESPIRATORY DISTRESS SYNDROME

(71) Applicant: ReversPAH LLC, Denver, CO (US)

(72) Inventors: Norbert F. VOELKEL, Denver, CO (US); Charles MAGOLSKE, Denver, CO (US)

(73) Assignee: ReversPAH LLC, Denver, CO (US)

(21) Appl. No.: 19/095,521

(22) Filed: Mar. 31, 2025

Related U.S. Application Data
(62) Division of application No. 17/398,156, filed on Aug. 10, 2021.
(60) Provisional application No. 63/063,528, filed on Aug. 10, 2020.

(51) Int. Cl. A61K 33/24; A61K 31/381; A61K 31/495; A61K 45/06

(52) U.S. Cl. CPC A61K 33/24; A61K 31/381; A61K 31/495; A61K 45/06

(57) ABSTRACT

A method of treating a Corona Virus, e.g., COVID 19, Influenza and ARDS, is provided. A copper chelator including a tetrathiomolybdate salt is administered with a 5-lipoxygenase enzyme inhibitor, e.g., Diethylcarbamazine or Zileuton. Baicalin, Bufalin, Quercetin, Curcumin, inhibitors of NF-kappaB, the Applied Therapeutics Aldose Reductase inhibitor AT-001, Sulforaphane or Fluvoxamine can be additional drugs. This is an intervention treatment of a Corona Virus, e.g., COVID 19, ideally in the second phase of the disease, in the Pulmonary Phase, preferably prior to the Hyper-Inflammation Phase, as a preventive therapy to reduce the need for a ventilator and increase the survival of hospitalized patients. The two-drug treatment combination aims at preventing ARDS and other organ damage caused by COVID 19 infection by targeting the intravascular disease component. Tetrathiomolybdate in oral and Intravenous forms combined with the other drugs in intravenous or inhaled forms are designed to treat advanced forms of these diseases.

### CROSS-REFERENCE TO RELATED APPLICATIONS

The present patent application is a U.S. Divisonal Application of U.S. Utility patent application Ser. No. 17/398,156, filed on Aug. 10, 2021 which Claims benefit of U.S. Provisional Patent Application No. 63/063,528, filed on Aug. 10, 2020, the disclosures of which are hereby incorporated by reference.

### FIELD

The present invention relates to methods and compositions for treating corona viruses, such as COVID-19, its analogues, progeny and variants, influenza, and acute respiratory distress syndrome.

### BACKGROUND

Currently there is no agreed standard of care for COVID-19 or new Corona viruses that may arrive in the future and many current developments focus on anti-viral activity, vaccination development with the idea that the virus spreading can be stopped. One cause of death during the course of the COVID-19 Corona Virus is Acute Respiratory Distress Syndrome (ARDS), which is the acute failure of the lungs to function as the gas exchanger. ARDS is not a disease, but a syndrome which has multiple causes and can be triggered by sepsis, trauma, a complication of surgery, mass transfusions and aspiration of gastric contents. ARDS is also caused by viral infections (Corona virus, Hanta virus, Herpes virus and Influenza virus). Outside of the current COVID-19 Corona Virus pandemic, ARDS in the USA has an incidence of 200,000 patients per year with a mortality rate of 22-33%. There is no effective treatment of fully developed ARDS, which is why when COVID-19 Corona Virus advances to the point creating ARDS and damage to organs other than the lung, death results. The present ARDS treatment consists of low volume mechanical ventilation and conservative management of intravenous fluids. The best chances to avoid the manifestation of ARDS are in the prevention of progression from sepsis or diffuse alveolar damage to fully developed life-threatening ARDS. In a number of patients ARDS is followed by kidney failure and sometimes multi-organ failure. There is a therapeutic window for early intervention, that is the administration of drugs to patients at risk for developing ARDS, because the median time for the development of ARDS is 2-7 days after hospitalization. For COVID-19 Corona Virus patients, the time window is often several days more. COVID-19 infected patients are at risk when they have fever, pulmonary infiltrates, and high plasma levels of C-reactive protein (CRP) and when they have co-morbidities like cardiovascular diseases or chronic lung diseases (25).

Research has illustrated that the COVID-19 Corona virus has three stages, as shown in FIG. 1 of the Drawings: Stage 1—Early Infection, Stage 2—Pulmonary Phase, and Stage 3—Hyper-Inflammation Phase. During Early Infection, patients have mild symptoms including fever, dry cough, fatigue, myalgias, headache dyspnea, and (in about 50% the patients) GI symptoms such as nausea vomiting or diarrhea. In the Pulmonary Phase patients experience of shortness of breath (dyspnea) with hypoxemia as well as abnormal infiltrates on chest imaging, transaminitis, low-normal procalcitonin. In the Hyper-Inflammation Phase patients experience ARDS, systemic inflammatory response syndrome (SIS) and multi organ dysfunction due to thromboembolic phenomena (kidneys, heart, liver, CNS). In this catastrophic phase inflammatory markers (IL-1, IL-6, and IL-8) are elevated, a troponin leak indicates myocardial damage and NT-proBNP elevation reflects myocardial insufficiency. The illness progresses and severity from Stage 1 to Stage 2 to Stage 3. In Stage 3, many of the patients die (25).

COVID-19 does not always result in severe sickness. In fact, it is estimated that a small percentage of those who contract the virus need to be hospitalized. For those that are hospitalized the percentage that die varies depending on the age of the patient and any pre-existing conditions. For those patients the progression to Stage 2 and Stage 3 of this disease, the probability of death is relatively high. The fatal organ manifestations of COVID-19 disease are by and large acute lung injury-with and without pulmonary hypertension (19) leading to respiratory failure and cardiovascular involvement leading to heart failure (Citations 1-5). Underlying both manifestations are inflammation driven by multiple cell/cell interactions and in situ thrombosis which is a consequence of inflammation of endothelial cells.

It is generally accepted that that with COVID-19-induced disease, the elderly, patients with comorbidities and immune compromised patients can develop severe disease and that death is due to lung-and heart failure and multi-organ failure. According to the study by Li et al (3) the most common organ damage outside the lung was heart damage. Although the precise mechanism of heart injury is not completely clear, an overwhelming immune inflammatory response and a cytokine storm are the most likely cause. Up to 8% of severely ill patients demonstrate a troponin leak that reflects myocardial tissue damage. In children, a Kawasaki-like inflammation of the vasculature has been recognized. While initially it had been found that Alveolar Type II cells and macrophages are infected by the COVID-19, now it is also known that endothelial cells are infected. Thrombotic manifestations have been recognized leading to acute coronary syndromes; myocarditis has also been described and fatal arrhythmias (1,2,4).

Common to heart and lung failure syndromes developed by COVID-19 is the inflamed endothelium (the endothelium can be considered an organ and it is noteworthy that the largest number of endothelial cells anywhere in the body is in the lungs) that becomes the staging ground for multi cell type conglomerates that clog vessels and capillaries (not just macrophages, but also platelets, neutrophils and red blood cells form these conglomerates). We define the pathobiologically critical mechanism of “intra-vascular inflammation” as the formation of multi-cellular aggregates adhering to inflamed endothelial cells.

Experimentally COVID-19-infected rhesus macaques developed acute lung injury that was characterized by large perivascular lymphocyte clusters and alveoli filled with macrophages and neutrophils (6).

A human study examined the bronchoalveolar lavage fluid (BALF) from COVID-19-infected patients with severe lung injury and reported the presence of multiple inflammatory cells, many of bone marrow origin, like myeloid dendritic cells, mast cells, plasma cells and T-lymphocytes. The authors describe a highly pro-inflammatory macrophage microenvironment and the presence of both M1 and M2 macrophages that express NF-kappaB and STAT 1 and STAT2 (7). NF-kappaB is a master transcription factor that is responsible for the transcription of a number of genes encoding inflammatory mediators.

Other recent articles discuss the roles of oxidant stress (8,9). In the context of intravascular inflammation, the production of reactive oxygen species is to be expected and their cell-injurious potential is appreciated. While it remains presently unresolved whether COVID-19-related lung damage is a special form of ARDS, there can be no doubt that inflammation, including intravascular inflammation is instrumental in causing organ damage and the demise of patients, is similar if not identical to ARDS. The recent report by Ackermann et al. (24) illustrates the vascular damage and micro-thrombi.

FIG. 3 of the Drawings illustrates the intravascular inflammatory environment. This FIG. 3 depicts cell-cell interactions within the lung vessels and likely also the coronary vessels and is likely applicable to the intravascular events occurring in severe COVID-19 disease. There are several pathways whereby sepsis induces injury to the endothelium. Sepsis upregulates the expression of selectins on the endothelium (P- and E-selectins), to which activated leukocytes (both neutrophils and monocytes) and platelet aggregates can adhere and induce an increase in endothelial permeability. The potential role of neutrophil extracellular traps (NETS) and histone release is also included as well as olfactomedin 4, lipocalin 2, and CD 24, and bacterial permeability increasing protein, products primarily of neutrophils. Some circulating factors in the plasma are both biomarkers of injury and also enhance the injury, including Ang-2 and VEGF. In addition, the diagram shows circulating factors that enhance inflammation such as IL-8 and IL-6, sTNFr-2. Markers of endothelial injury also include vWF and sFLT-1, the circulating VEGF receptor. Also noted in the diagram are components of the activated protein C complex including protein C, protein S, factor V, and thrombomodulin because sepsis deranges the normal function of activated protein C leading to a pro-coagulant environment.

The corona virus infection (COVID-19) can become lethal because of inflammatory organ damage: in the lung leading to diffuse alveolar damage (DAD) and thrombotic vascular occlusion and ARDS, and in the heart, another organ that is attacked, via myocarditis and heart muscle damage (3-5). There is a consensus that the inflammatory response triggered by the COVID-19 determines the outcome.

Influenza virus infections are an annual occurrence which result in a considerable global mortality. Vaccinations are developed every year to fight new strains of influenza viruses. There is no effective treatment of patients that are immunosuppressed or of children that, when infected have a high mortality, as do the elderly.

The role of the immune system as a defender against inflammatory organ damage is appreciated. There is also a consensus that cytokines play an important part in the manifestation of the inflammatory response; of particular interest are IL-1, IL-6 and IL-8, and therapeutic blockade of their receptors are strategies that are actively being investigated in COVID-19-infected patients.

These cytokines can be produced by several inflammatory cells, but also by endothelial cells and by vascular smooth muscle cells. Central in the generation of these cytokines and also of TNF alpha is the transcription factor NF-kappaB and IL-1 can activate IL-6 production leading to a vicious cycle of enhanced cytokine production.

Both the TNF alpha-induced as well as the miR-125b-induced activation of NF-kappaB are copper dependent and copper chelation has shown to inhibit NF-kappaB activation in various cell types, including endothelial cells which likely develop an inflammatory phenotype (one characteristic of which is NF-kappaB expression). In addition, Toll Like Receptor (TLR) activation causes the up-regulated expression of several copper transporters, in particular Ctr1, Ctr2 and ATP7A (15).

At present, there are no drugs for COVID-19 treatment that will prevent the progression of the coronavirus and prevent intravascular inflammatory and procoagulant mechanisms that pave the way to lung damage and heart failure.

At present, there are no drugs for Influenza treatment that will prevent the progression of the Influenza virus and prevent intravascular inflammatory and procoagulant mechanisms that pave the way to lung damage and heart failure.

### SUMMARY

In general terms, the present invention is based on learning that fatal events from a Corona Virus, Influenza or ARDS can be prevented by drugs that interfere with intra-vascular inflammation. See FIGS. 3, 6 and 13 of the Drawings. The intravascular events tie together lung and heart failure. Briefly, the “sick lung circulation” releases a myriad of mediators that enter the next proximate circulation: the coronary circulation. The “bad humor” released by the sick lung circulation spills over into the systemic circulation and also reaches the central nervous system. The overall concept is that the injured lung—in particular the lung vessels—emits signals of cell damage; these signals include chemotactic factors such as chemokines and leukotrienes, cell fragments and free DNA.

Specifically, the present invention is based first on the understanding of the sick lung circulation, whereby the microvasculature of the lung is gravely affected in COVID-19 lung injury (see FIG. 5, Ref.24). It is undergoing an intravascular inflammatory reaction that produces mediators (by the endothelial cells, EC, that have been shown to be infected with the COVID-19 virus particles) and by multiple cell-cell interactions. FIG. 4—illustrates this “bad lung humor” concept. The lung has the largest capillary network of the human body and thus the largest number of endothelial cells (EC). COVID-19-infected EC become cells that participate in inflammatory cell-cell interactions and produce injurious mediators that spill out from the “sick lung circulation”. FIG. 5 illustrates how airborne avenues lead to heart and lung damage.

The present invention is based on in part the learning by the inventors that the micro-vessels with their inflammation and thrombotic obliterations (FIG. 6) are a significant component of COVID-19 severe disease—and ARDS, and for this reason, the vascular disease manifestations are a treatment target. For involvement of the enzyme 5-lipoxygenase (5-LO) (see FIGS. 11 and 12).

The inventors are employing strategies that (1) inhibit chemotaxis of inflammatory cells into the lung and the heart, (2) decrease vascular permeability and leak (3) decrease the activity of the master inflammatory mediator transcription factor NF-kappaB (7,8,16,18), (4) Decrease VEGF production and action and (5) inhibit or retard the virus entry into the cells.

The specific hypothesis is that a 5-lipoxygenase inhibitor and antioxidant—such as Diethylcarbamazine (DEC) or Zileuton, together with tetrathiomolybdate (TTM), a copper chelator with anti-inflammatory properties and also an antioxidant and inhibitor of VEGF production to inhibit the intravascular inflammatory and procoagulant mechanisms that pave the way to lung damage and heart failure. The two drugs act by different mechanisms, a 5-lipoxygenase inhibitor and antioxidant, such as DEC or Zileuton, by inhibiting the formation of leukotriene B4 inhibits the chemotaxis of neutrophils and macrophages into the injured lung and also endothelial cell damage. TTM may have pleiotropic actions that include inhibition of viral entry into cells, and inhibition of VEGF-triggered vascular leak (VEGF is a vascular permeability-enhancing factor). This invention is to use the combination of two drugs with different mechanisms of action (a 5-lipoxygenase inhibitor like DEC or Zileuton) plus TTM as primary drivers to prevent disease progression and also to treat this disease. Due to the safety of these drugs, they can be used along with other treatments. Further, the inventors include as an invention other drugs that can be used in concert with these two core drugs, such as anti-inflammatory antidepressants (for example, Selective Serotonin Reuptake Inhibitors (SSRIs), (such as Fluvoxamine, and Apigenin), Indole-3-carbinol (i3c), Bufalin, Baicalin, Curcumin, Quercetin, the Applied Therapeutics Aldose Reductase inhibitor AT-001, which has antioxidant properties, and an antiviral or corona virus antibody medications that may be available. Further, therapy is further enhanced with the addition of Prostacyclin Analogues, such as Iloprost, and their PGI₂ Receptor Agonist Selexipag, Treprostenil, and Beraprost.

This invention pursues the strategic goals to protect the lung and the cardiovascular system from developing organ damage by employing the two core drugs (above) and more existing drugs that achieve these goals. The primary two drugs are TTM and either DEC or Zileuton. Additional drugs can include SSRIs, such as the antidepressant Fluvoxamine, which has been shown to reduce inflammation via stimulation of the Sigma-1 receptor, and/or Ivermectin, which has been shown to inhibit the in vitro replication of the COVID-19 virus. Caly, L. et al. published that Ivermectin inhibits the replication of SARS-CoV-2 in vitro (Antivir Res, Apr. 3, 2020) and also Curcumin that reduces inflammation. As these drugs are safe this battery of drugs can be used with other treatments, such as oxygen, nitrous oxide, and steroids, as such treatments that on their own cannot prevent intravascular inflammatory and procoagulant mechanisms that pave the way to lung damage and heart failure.

The present invention recognizes that the corona virus infection (COVID-19) and Influenza can become lethal because of inflammatory organ damage of the lung leading to diffuse alveolar damage (DAD) and thrombotic vascular occlusion and ARDS, and in the heart, another organ that is attacked, via myocarditis and coronary syndromes leading to heart muscle damage; unknown mechanisms, including cytokine-dependent mechanisms can damage the heart muscle directly (3-5). This learning and the understanding that inflammatory response triggered by COVID-19 and Influenza has allowed the inventors to determine that a combination of drugs, specifically TTM plus DEC or Zileuton, will address key biological functions that create the events that lead to patients being put on ventilators, developing organ damage, and leading to death. The inventor's knowledge of the mechanism of action of these drugs, the specific inhibitory activities they will generate, allows the inventors to also in concert with either TTM or DEC or both add other co-drugs, such as the SSRI anti-inflammatory antidepressant Fluvoxamine, Sulforaphane, Ivermectin and Curcumin and variants thereof. These same drugs are designed to treat ARDS caused by conditions other than a Corona virus. (with the exception of Ivermectin which has likely no utility in ARDS).

The role of the immune system as a defender against inflammatory organ damage is appreciated. There is also a consensus that cytokines play an important part in the manifestation of the inflammatory response; of particular interest are IL-1 and IL-6, and therapeutic blockade of their receptors are strategies that are actively being investigated in COVID-19 infected patients. Both cytokines can be produced by several inflammatory cells, but also by endothelial cells and by vascular smooth muscle cells. Central in the generation of these cytokines and also of TNF alpha is the transcription factor NF-kappaB.

Both the TNF alpha-induced as well as the miR-125b-induced activation of NF-kappaB are copper dependent (16,18) and copper chelation has shown to inhibit NF-kappaB activation in various cell types, including endothelial cells which likely develop an inflammatory phenotype (one characteristic of which is NF-kappaB expression). In addition, Toll Like Receptor (TLR) activation causes the up-regulated expression of several copper transporters, in particular Ctr1, Ctr2 and ATP7A (15).

The transcriptional activity of HIF-1 alpha is also copper dependent and consequently the production of VEGF, originally named “vascular permeability factor. VEGF does play a role in ARDS as a vascular permeability (leak) enhancing factor.

In conclusion: copper chelation by TTM, in the context of COVID-19 and Influenza triggered inflammation, will inhibit cytokine production and HIF-1alpha-dependent gene transcription. The latter is of importance because of the tissue hypoxia of the damaged organs.

In one embodiment of the invention, the inventors are combining the use of a 5-lipoxygenase inhibitor like DEC or Zileuton in the treatment of a subject suffering from COVID-19, a mutation of COVID-19, another Corona Virus with similar mechanisms of action to COVID-19 or Influenza with TTM as the mechanisms of action including the inhibition of chemotaxis and inhibition of vascular leak—note that one of the most powerful chemotactic mediators is leukotriene B4 (LTB4), a product of activated 5-lipoxigenase. LTB4 is produced by macrophages, eosinophils, neutrophils cooperating with erythrocytes and by activated endothelial cells. The specific hypothesis here is that LTB4 is of critical importance in the development of organ failure due activation of chemotaxis and direct damage to the endothelium resulting in vascular leakage. DEC or Zileuton are expected to inhibit the synthesis of LTB4, but in addition the synthesis of LTC4, a peptido-leukotriene that is vaso-and bronchospastic.

DEC has antioxidant properties and inhibits oxidant stress involved in COVID-19 inflammation (8,9), as well as Influenza. Inhibition of inflammatory mediator production and DEC may inhibit 5-lipoxygenase-dependent activation of NF-kappaB.

The present inventors also discovered that TTM treatment, that is treatment with a copper chelator, inhibits NF-kappaB activation in various cell types, including endothelial cells which likely develop an inflammatory phenotype, can be further enhanced by the combination of the copper chelator comprising the TTM salt and at least one active agent such as Diethylcarbamazine. The copper chelator comprising the TTM ammonium salt and at least one active agent may be administered separately or together in a combined pill. For example, the copper chelator comprising the TTM ammonium salt may be administered orally and the at least one active agent may be administered intravenously or orally.

In one embodiment, the present invention also provides a composition comprising effective amounts of the copper chelator comprising a TTM salt and a 5-lipoxygenase inhibitor such as DEC or Zileuton. In addition to the combination of TTM and DEC or TTM and Zileuton, other active agents may be Ivermectin an anti-parasite agent that has been shown in in vitro studies to inhibit entry of the COVID-19 Corona virus into cells, Apigenin, Indole-3-carbinol, Bufalin, Baicalin, Curcumin (Quercetin), an Aldose Reductase inhibitor, and the anti-inflammatory antidepressant Fluvoxamine or Sulforaphane. Such compositions may be in an intravenous form or an oral form, such as a tablet, a microtablet, or a capsule. The oral forms may provide a delayed release of the TTM salt after passage through the stomach. Such a composition may release, for example: (1) a TTM salt after the oral form of TTM passes the stomach and (2) at least one other active agent released in the stomach or after the other active agent passes the stomach.

The summary mechanisms of action TTM will accomplish include inhibition of chemotaxis, inhibition of vascular permeability, inhibition of inflammatory mediator production, inhibition of activation of the master transcription factor NF-kappaB and the decrease of VEGF production and to a degree inhibition of the virus entry into the cells. VEGF is a powerful factor that mobilizes precursor cells from the bone marrow. A role for VEGF in ARDS has been acknowledged as vascular permeability factor. VEGF is 50 times more effective than histamine to cause vascular leak. The HIF-1alpha-dependent transcription of the VEGF gene is also copper-dependent and thus TTM decreases VEGF production.

The summary mechanisms of action for an DEC are inhibition of the enzyme 5-lipoxygenase, inhibition of oxidants and inhibition of NF-kappaB-dependent gene transcription. In the aggregate—by such molecular mechanisms—DEC inhibits chemotaxis and preserves normal endothelial cell function.

The principal mechanism of action for the anti-inflammatory drug Sulforaphane is the activation of the transcription factor Nrf2. This transcription factor is a switchboard that transcribes a host of antioxidant enzyme genes-resulting in the production of antioxidant enzymes, such as superoxide dismutase and catalase. Because inflammation is associated with oxidant stress, Sulforaphane reduces the oxidant stress component of inflammation.

The principal mechanism for the anti-inflammatory action of the antidepressant and antianxiety drug Fluvoxamine is the stimulation of the endoplasmatic reticulum Sigma-1 receptor which restricts Inositol Requiring Enzyme 1 [IRE 1]-dependent activation of inflammatory mediators.

The direct administration of the two drugs, Diethylcarbamazine and a Prostacyclin Analogue, such as Beraprost, to the lung, via inhalation of the nebulized powder, has the advantage of delivering a relatively high dose of the drugs to the lung—without any spill-over into the peripheral systemic circulation. The drug's action is restricted to the target: the airways and the lung tissue. The benefit is the delivery of a pulmonary vasodilator and an inhibitor of the 5-lipoxygenase. Both drugs are synergistic in inhibiting the inflammation in the lung without any toxicity or side effects.

### BRIEF DESCRIPTION OF THE FIGURES

Advantages of embodiments of the present invention will be apparent from the following detailed description of the exemplary embodiments. The following detailed description should be considered in conjunction with the accompanying figures in which:

FIG. 1 illustrates the three stages of the COVID-19 Corona virus: Stage 1—Early Infection, Stage 2—Pulmonary Phase, and Stage 3—Hyper-Inflammation Phase;

FIG. 2 shows the structure of an exemplary TTM salt of the present invention, specifically the ammonium salt of TTM, or ATTM;

FIG. 3 illustrates the intravascular inflammatory environment. This figure depicts cell-cell interactions within the lung vessels and likely also the coronary vessels and is likely applicable to the intravascular events occurring in severe COVID-19 disease.

FIG. 4 is an illustration of the Sick Lung circulation and how it affects the heart.

FIG. 5 is an illustration of the progress how an airborne Corona Virus enters the lungs and then moves to the heart and heart failure, as cited in Geng Y-J. et al. Cardiovasc Pathol, Apr. 17, 2020.

FIG. 6 illustrates the inflammation and the destruction of the endothelial cells, the creation of microthrombi and with the destruction of the endothelial cells the red blood cells leaking out of the alveolar capillaries. Slightly expanded alveolar walls with multiple fibrinous microthrombi are indicated by arrowheads.

FIG. 7 illustrates how both ligation of the TNF alpha receptor and the action of copper via mRNA 125b activate NF-kappaB, and thus transcription of NF-kappaB-dependent genes encoding proteins involved in inflammation.

FIG. 8a illustrates both the transcription factor NF-kappaB and copper transport into the cell (15) and within the cell can be inhibited with the copper chelator TTM. TTM would be expected to reduce the generation of cytokines in different COVID-19-Infected cell types. Toll-like receptor activation of cells increase the expression of genes encoding copper transporters—which in turn facilitate copper-dependent activation of NF-kappaB, a likely scenario occurring in COVI-19 triggered intra-vascular inflammation.

FIG. 8b illustrates another factor the inventors have considered is that the phenotypical shift of macrophages to the pro-inflammatory M1 cell type is copper-dependent. It can be postulated that the so-called cytokine storm observed clinically in sick COVID-19-infected patients is due to interactions of multiple professional inflammatory cells and activated structural cells. Cell-cell interactions in the infected lung are of critical importance. As far as the production of leukotrienes is concerned, research has shown that red blood cells can donate an enzyme to neutrophils and that this results in a potentiated leukotriene B4 production. This concept of ‘transcellular metabolism’ has been widely accepted.

FIG. 9 illustrates that DEC treatment significantly inhibited neutrophil infiltration. The figure is reproduced from Ribeiro et al. (23). The authors show in a mouse acute lung injury model that DEC pretreatment prevented the influx of neutrophils into the lung, using the neutrophil and macrophage marker myeloperoxidase.

FIG. 10 illustrates the effect of DEC on carrageenan-induced TNF-alpha and nitric oxide production in the lung. The figure is reproduced from Ribeiro et al. (23). (a) shows TNF-alpha levels were significantly elevated 4 hours after carrageenan administration in the CAR group in comparison to the sham group. DEC significantly reduced the TNF-alpha levels, but INDO did not reduce the TNF-alpha level in comparison to the CAR group. (b) shows that nitrite and nitrate levels, stable NO metabolites, were significantly increased in the pleural exudates 4 hours after carrageenan administration in comparison to the sham group, and DEC and INDO significantly reduced the nitrite and nitrate level in the exudates. Data expressed as means +/− S. E. M. from n=8 mice for each group *p<0.05 versus carrageenan. This pre-treatment inhibits the inflammation in the lung as shown by reduction in TNF-alpha and Nitric oxide production.

FIG. 11 illustrates the inhibition of 5-lipoxygenase by DEC (20). In addition to the canonical action of the 5-LO enzyme which is the synthesis of the inflammatory leukotrienes, there is also the additional property of 5-LO to increase in the nucleus NF-kappaB-dependent gene transcription. A vastly enhanced expression and activation of the 5-LO enzyme can be postulated to occur in the COVID-19-triggered intravascular inflammation.

FIG. 12 illustrates the 5-LO dependent expression signature and that 5-LO may work as a transcription factor IL-1beta, IL-6, BC12, ET, beta catenin, cmyc. IL-1 and IL-6 are involved in COVID-19 infection and organ damage.

FIG. 13 illustrates an example of the cell aggregates filling the injured lung vessel. This is an electron micrograph that shows what is in the lung vessels during the development of lung injury. Cell aggregates—including red blood cells are also playing, because they can participate in making inflammatory mediators—like leukotrienes.

FIG. 14 illustrates how Ctr1 and ATP7A are important for viral replication [Rupp JC et al, Virol. J, 2017]. This paper titled “Host Cell Copper Transporters CTR1 and ATP7A Are Important for Influenza A Virus Replication” teaches that chelating copper, resulted in moderate defects in viral growth. RNAi knockdown of the high-affinity copper importer CTR1 resulted in significant viral growth defects (7.3-fold reduced titer at 24 hours post-infection, p=0.04). Knockdown of CTR1 or the trans-Golgi copper transporter ATP7A significantly reduced polymerase activity in a minigenome assay. The Figure also illustrates copper-mediated regulation of the influenza virus life cycle. Extracellular copper [Cu²⁺] shares topological space with virion binding to host cell, and viral entry steps within the endosome. CTR1 imports extracellular copper to the cytoplasm. Intracellular copper [Cu¹⁺] is associated with the ATOX1 chaperone and other metalloproteins. From there, copper is actively transported into the secretory pathway by ATP7A. ATP7A plays a role determining copper concentration in the cytosol and in ER, Golgi, and other membrane bound compartments, where the viral glycoproteins HA and NA (o) are synthesized and mature. New viral RNA is synthesized in the nucleus, where ATOX1 may transport intracellular [Cu¹⁺]. In complex with matrix proteins (M1 and M2), genomic viral RNA progeny is exported from the nucleus to associate with M1, M2, HA, NA, and other proteins to assemble budding virions at the plasma membrane, a site that is topologically in the cytosol.

FIG. 15 illustrates the copper Proteome. The Host Cell Copper Transporters CTR1 and ATP7A are important for influenza A Virus Replication and may also be important for COVID 19. [The copper proteome; Blockhuys S.et al, 2017]. The protein ATOX1 is a copper chaperone which transports copper from Ctr1 to ATP7A and ATP7B. ATOX1 also controls cell proliferation. Copper chelation inhibits this intracellular transport—and because copper levels are elevated in inflamed tissues TTM has also anti-inflammatory actions.

FIG. 16 Illustrates the proteomic VEGF-A signaling pathways data during hypoxia in BM-EPCs suggest an important role for canonical VEGF-A signaling, regulation of redox homeostasis, cell survival, cell migration and inflammation.

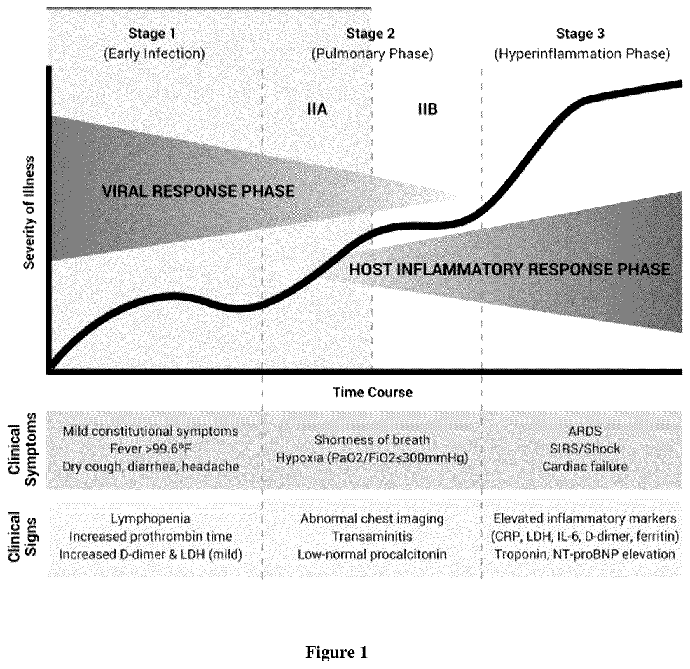

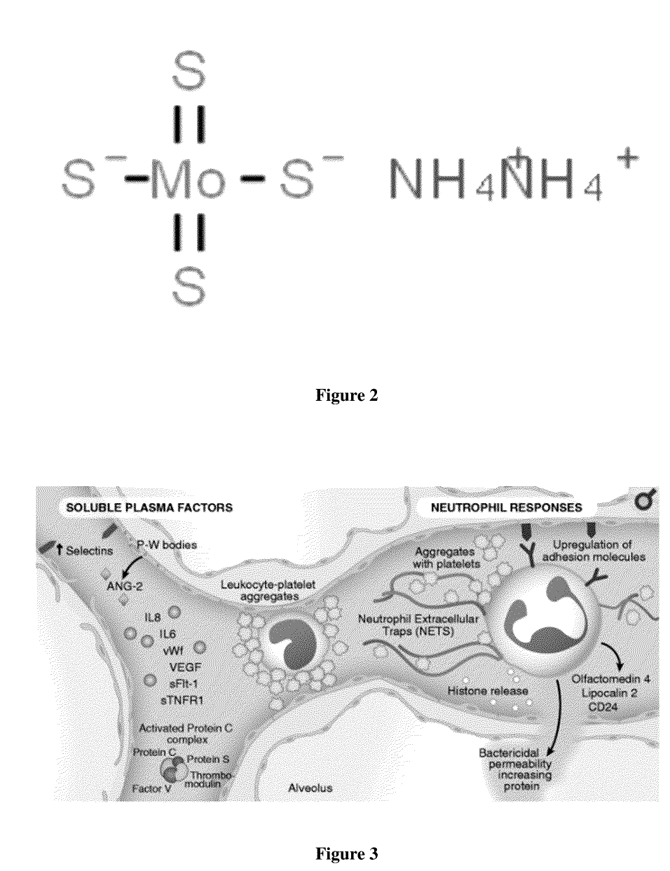

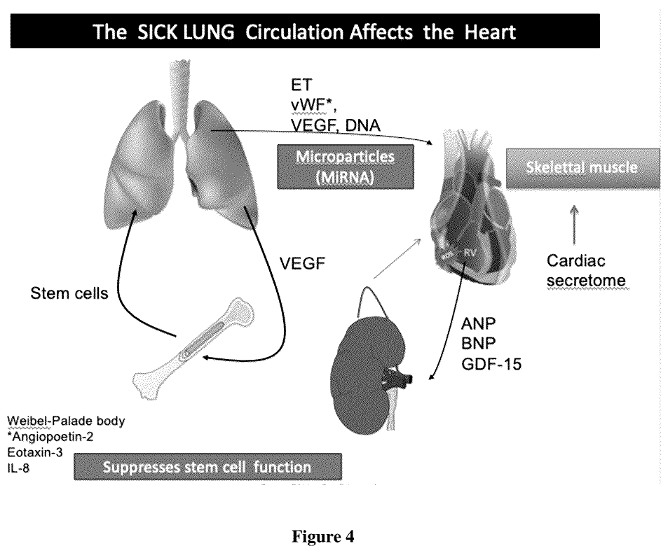

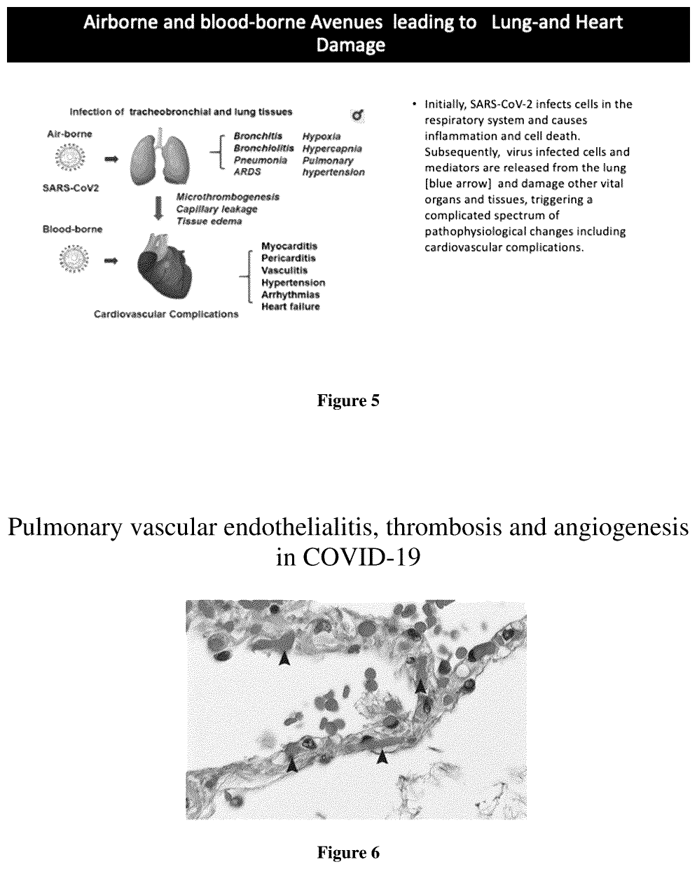

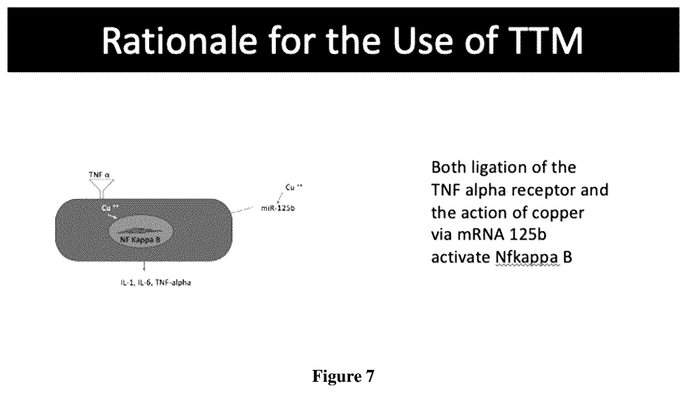

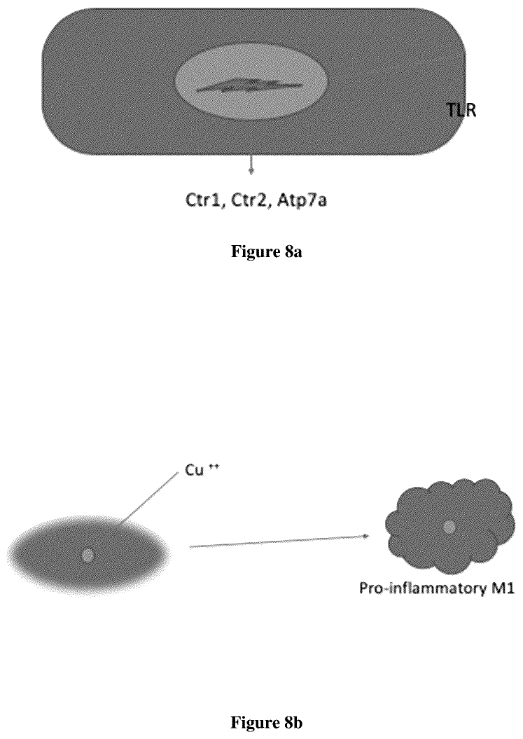

### DETAILED DESCRIPTION

Aspects of the present invention are disclosed in the following description directed to specific embodiments of the invention. Those skilled in the art will recognize that alternate embodiments may be devised without departing from the spirit or the scope of the claims. Additionally, well-known elements of exemplary embodiments of the invention will not be described in detail or will be omitted so as not to obscure the relevant details of the invention. Further, to facilitate an understanding of the description discussion of several terms used herein follows.

As used herein, the word “exemplary” means “serving as an example, instance or illustration.” The embodiments described herein are not limiting, but rather are exemplary only. It should be understood that the described embodiments are not necessarily to be construed as preferred or advantageous over other embodiments. Moreover, the terms “embodiments of the invention”, “embodiments” or “invention” do not require that all embodiments of the invention include the discussed feature, advantage, or mode of operation.

Copper, due to its Fenton Chemistry, serves as an important cofactor for numerous proteins and enzymes involved in both physiologic and pathological process. The proteins are secreted, intracellular or transmembraneous. There are more than 50 copper-binding proteins in the various compartments of a cell (membrane, cytoplasm, nucleus, and mitochondria) they function as copper transporters, chaperones, and enzymes. In theory all of these copper-binding proteins may be affected to various degrees by the copper chelator TTM.

The present invention is based on the discovery that high levels of extracellular and intracellular copper play a critically important role in intra-vascular inflammation, specifically via inhibition of the vascular permeability factor VEGF and inhibition of NF-kappaB-dependent gene transcription.

The present inventors understood in order to prevent patients infected with a Corona Virus, such as COVID-19, a mutation of COVID-19, or another Corona Virus with similar mechanisms of action to COVID-19, from progressing to the need to be treated by use of a ventilator and possible death, administration of drugs that inhibit chemotaxis and vascular leak and endothelial cell damage and prevent progression to hyperinflammation and ARDS must be selected. Such drugs must intervene early enough to prevent disease progression and also treat this disease and protect the lung and the cardiovascular system from developing organ damage. To do so, they realized the mechanisms of action these drugs had to provide is as follows:
(i) Inhibit chemotaxis of inflammatory cells into the lung and the heart.
(ii) Decrease VEGF production and action.
(iii) Decrease vascular permeability and leak.
(iv) Decrease the activity of the master inflammatory mediator transcription factor NF-kappaB activation in various cell types, including endothelial cells which likely develop an inflammatory phenotype (one characteristic of which is NF-kappaB expression).
(v) Inhibit or retard the virus entry into the cells.
(vi) Provide a treatment that has anti-inflammatory properties and also an antioxidant and inhibitor of VEGF production to inhibit the intravascular inflammatory and procoagulant mechanisms that pave the way to lung damage and heart failure.
(vii) Address LTB4, as it is of critical importance in the development of organ failure due activation of chemotaxis and direct damage to the endothelium resulting in vascular leakage and in addition to the synthesis of LTC4, address peptido-leukotriene that is vaso-and bronchospastic.
(viii) Inhibit the formation of leukotriene B4 that then inhibits the chemotaxis of neutrophils and macrophages into the injured lung and also endothelial cell damage.
(ix) Address cytokines that play an important part in the manifestation of the inflammatory response; of particular interest are IL-1, IL-6, and IL-8.
(x) Inhibit the activity of the important hypoxia-induced transcription factors HIF-1alpha and HIF-2 alpha.
(xi) Stimulating the endoplasmatic reticulum [ER] Sigma-1 receptor which dampens inflammation.

It follows during treatment of COVID-19 patients, as well as those suffering from a mutation of COVID-19 or another Corona Virus with similar mechanisms of action to COVID-19, with copper chelator comprising the TTM salt would provide some of the mechanisms of action needed.

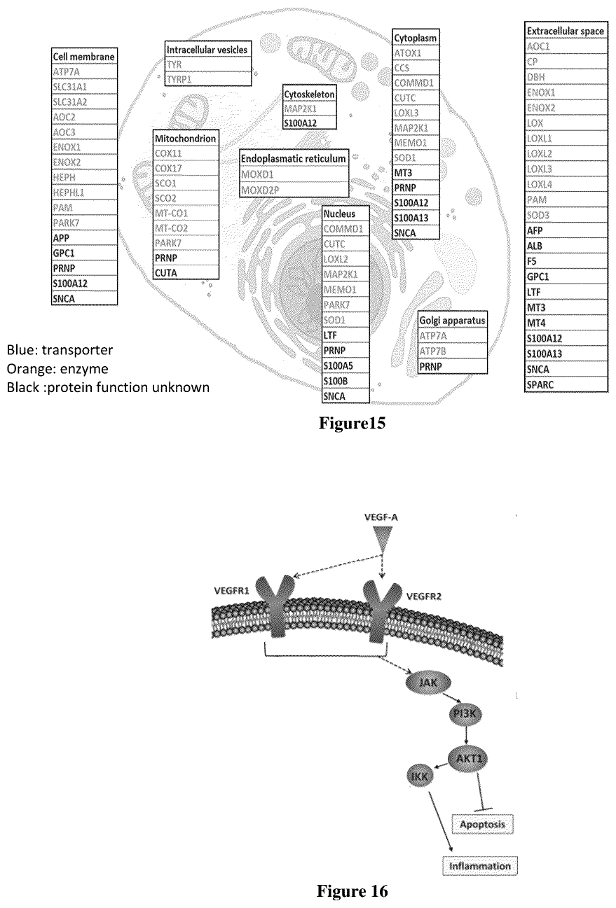

The proposed mechanism of action of the copper chelator comprising the TTM salt in COVID-19 patients, as well as those suffering from a mutation of COVID-19 or another Corona Virus with similar mechanisms of action to COVID-19, is several fold: reduction of vascular cell inflammation, and reduction of chemotaxis of inflammatory cells and the transport from the bone marrow into the lung. VEGF is a powerful factor that mobilizes precursor cells from the bone marrow which may participate in the injury and repair process. By inhibiting the VEGF gene transcription TTM will decrease the VEGF-dependent vascular permeability increase and angiogenic changes that are sequelae of the intravascular inflammation. FIG. 16.

Based on this research and trials for other indications using TTM, it is hypothesized by the present inventors that treatment with the copper chelator comprising a TTM salt will provide the required mechanisms of action and a 5-lipoxygenase inhibitor and antioxidant—such as Diethylcarbamazine (DEC) or Zileuton will provide the other required mechanisms of action. Further, the inventors have identified other drugs that can be administered to assist in creating the required and desired mechanisms of action.

The copper chelation has also been shown in culture studies of infected lung cells to significantly reduce the viral replication rate. TTM has the characteristic of chelating copper from the body. Influenza viruses not only replicate in the airway and lung tissue cells, but they also destroy these cells and cause in severe cases a pneumonia and acute lung injury [ARDS]. To decrease and significantly inhibit this inflammatory response is a treatment goal that cannot be achieved with antibiotics or with steroid drugs and is achieved with TTM.

The lung vessels and the capillaries are involved in this inflammation, caused by Influenza, that ultimately leads to a vascular leak and edema impairs the gas exchange function of the lung. While TTM inhibits influenza A viral replication the drug Diethylcarbamazine [DEC] inhibits chemotaxis of inflammatory cells (neutrophils, macrophages, and immune cells) into the lung vessels—principally via inhibiting the synthesis of leukotrienes [LTC4—which causes broncho- and vaso constriction and of LTB4 which causes lung endothelial cell injury.

TTM is effective for treating Influenza because it also inhibits the action of the master transcription factor NF-kappaB which is responsible for the activation of the genes that encode many cytokines (for example IL-1 and IL-6) and mediators of inflammation like TN alpha. TTM also inhibits the transcription factor HIF-1alpha that is responsible for the transcription of the VEGF gene, VEGF is a potent vascular permeability enhancer—known to play a pathognomonic role in ARDS.

Taken together: the combination of TTM+DEC has an anti-viral mechanism of action and inhibits pulmonary intravascular inflammation on several cellular and molecular levels. Therefore TTM+DEC are expected to have an impact as an effective treatment of influenza A disease with the benefit of being virus-strain independent.

Also, the present inventors determined that copper levels influence vascular inflammation. This discovery is based on the identification of four copper-dependent mechanisms.

First, copper is involved in stabilizing the ubiquitous transcription factor protein hypoxia-inducible factor 1-alpha (or HIF-1-α). HIF-1-α is responsible for the transcription of more than 100 genes, among them the genes encoding the angiogenic vascular endothelial growth factor (VEGF) and its kinase insert domain receptor (KDR). HIF-1-α induces the recruitment of bone marrow-derived vascular modulatory cells to regulate tumor angiogenesis and invasion Hoffmann BR. Et al.Physiol Genomics,2013 (27)

Both VEGF and its receptor play an important role in causing increased vascular permeability.

Second, copper plays a role in inflammation It has long been appreciated that the lung vascular lesions in IPAH are infiltrated by inflammatory and immune cells (Tuder et al., Am J Pathol, 1994 February; 144(2): 275-85.) These cells secrete mediators of inflammation, so-called cytokines—specifically the interleukins IL-1 and IL-6 (Humbert et al., Am J Respir Crit Care Med., 1995 May; 151(5):1628-31). In several cell- and organ systems it has been shown that a specific copper chelator, TTM salt, reduces the secretion of cytokines, as the TTM ammonium salt has an anti-inflammatory action in addition to the anti-angiogenic action.

Third, copper plays a role in the alteration of genes of the cytochrome P450. The lung and, in particular, the lung vascular endothelial cells (EC) are involved in drug metabolism and the handling of toxic substances. It is known that cigarette smoke toxins highly up-regulate the expression of specific drug metabolizing genes, the genes of the cytochrome P450 super-family. There are 56 known cytochrome P450 genes coding for 56 isozymes. These enzymes metabolize 75% of all drugs in use, including all of the vasodilator drugs conventionally used for PAH treatment. These enzymes also are involved in cell growth and differentiation, in cholesterol and estrogen metabolism and for many years a role of these enzymes in the pathogenesis of cancers has been examined (in particular prostate, breast and lung cancer) (Kwapiszewska G et al, Circulation Research, submitted 2019). Copper, however, has been shown to cause liver and kidney injury attributed to alterations of cytochrome P450 enzyme activities, and copper chelation has been demonstrated to protect against liver and kidney injury by inhibiting cytochrome P450 gene alterations.

Fourth, copper plays a role in angiogenesis, which can be a sequela of the intravascular inflammation. As in cancer, vascular cells can undergo a phenotype switch, which can be copper-dependent. Thus, a copper chelator would provide anti-angiogenic action and preserve the normal vascular cell phenotype.

One of the present inventors, Norbert F. Voelkel, determined that these four copper-dependent mechanisms involved in cell growth and differentiation, angiogenesis and inflammation are amenable to modification by treatment with a copper chelator comprising a TTM salt. Because of the potential for modifying any or each of these disease-contributing mechanisms the use of a copper chelator is proposed by Norbert F. Voelkel to treat intravascular inflammation.

In this context of this patent application, “abnormal copper handling” by the abnormally growing cells means and includes that there are potentially multiple and diverse reasons for the faulty handling of copper. There may be inherited or acquired mutations in the genes encoding copper transporters or copper binding proteins or mutations of one or several genes encoding cytochrome P450 enzymes causing abnormal copper handling and abnormal cellular metabolism.

The copper chelator comprises a salt of TTM, which is a highly effective copper-chelator for the purpose of the present invention. The salt may be according to formula I:

X(MoS₄)

X is (2Li)⁺², (2K)⁺², (2Na)⁺², Mg⁺², Ca⁺², or {[N⁺(R¹)(R²)(R³)(R⁴)][N⁺(R⁵)(R⁶) (R⁷)(R⁸)]};

R¹, R², R³, R⁵, R⁶, and R⁷ are independently H, or optionally substituted group selected from the group consisting of alkyl, alkenyl, alkynyl, aryl, heteroaryl, cycloalkyl, heterocycloalkyl, aralkyl, alkylaralkyl, heteroaralkyl, cycloalkyl alkyl, and heterocycloalkyl alkyl; and

R⁴ and R⁸ are absent or independently H, or optionally substituted group selected from the group consisting of alkyl, alkenyl, alkynyl, aryl, heteroaryl, cycloalkyl, heterocycloalkyl, aralkyl, alkylaralkyl, heteroaralkyl, cycloalkyl alkyl, and heterocycloalkyl alkyl;
wherein when R⁴ is absent, R¹ and R² together with N forms an optionally substituted 5- or 6-membered aromatic ring, wherein up to 2 carbon atoms in the ring may be replaced with a heteroatom selected from the group consisting of O, N, and S;
wherein when R⁸ is absent, R⁵ and R⁶ together with N forms an optionally substituted 5- or 6-membered aromatic ring, wherein up to 2 carbon atoms in the ring may be replaced with a heteroatom selected from the group consisting of O, NH, and S;
wherein R¹ and R², R² and R³, or R³ and R⁴, together with N optionally forms an optionally substituted cyclic structure;
wherein R⁵ and R⁶, R⁶ and R⁷, or R⁷ and R⁸, together with N optionally forms an optionally substituted cyclic structure;
wherein R⁴ and R⁸ may be joined by a covalent bond;
wherein R¹, R², R³, R⁵, R⁶ and R⁷ are each independently optionally substituted with one or more OH, oxo, alkyl, alkenyl, alkynyl, NH₂, NHR⁹, N(R⁹)₂, —C═N(OH), or OPO₃H₂, wherein R⁹ is each independently alkyl or —C(═O)(O)-alkyl;
wherein R⁴ and R⁸ are each independently optionally substituted with one or more OH, oxo, alkyl, alkenyl, alkynyl, NH₂, NHR⁹, N(R⁹)₂, —C═N(OH), or —⁺(R¹⁰)₃, wherein R¹⁰ is each independently optionally substituted alkyl; and
wherein one or more —CH₂— groups in R¹, R², R³, R⁴, R⁵, R⁶, R⁷ and R⁸ may be replaced with a moiety selected from the group consisting of O, NH, S, S(O), and S(O)₂.

In an exemplary embodiment, X is {[N⁺(R¹)(R²)(R³)(R⁴)][N⁺(R⁵)(R⁶)(R⁷)(R⁸)]} according to formula (II):

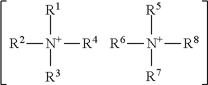

In one embodiment where X is {[N⁺(R¹)(R²)(R³)(R⁴)][N⁺(R⁵) (R⁶)(R⁷)(R⁸)]}, [N⁺(R¹)(R²)(R³)(R⁴)] and [N⁺(R⁵) (R⁶)(R⁷)(R⁸)] are the same or different.

In one embodiment where X is {[N⁺(R¹)(R²)(R³)(R⁴)][N⁺(R⁵) (R⁶)(R⁷)(R⁸)]}, R¹, R², R³, R⁵, R⁶, and R⁷ are independently H or C₁-C₁₀ alkyl. In another embodiment where X is {[N⁺(R¹)(R²)(R³)(R⁴)][N⁺(R⁵)(R⁶)(R⁷)(R⁸)]}, R¹, R², R³, R⁵, R⁶, and R⁷ are independently H, C₁-C₃ alkyl or C₁-C₆ alkyl. In a further embodiment where X is {[N⁺(R¹)(R²)(R³)(R⁴)][N⁺(R⁵)(R⁶)(R⁷)(R⁸)]}, R⁴ and R⁸ are independently H or C₁-C₆ alkyl.

In one embodiment where X is {[N⁺(R¹)(R²)(R³)(R⁴)][N⁺(R⁵)(R⁶) (R⁷)(R⁸)]}, R¹, R², R³, R⁵, R⁶, and R⁷ are independently H, methyl, ethyl, or propyl. In a further embodiment where X is {[N⁺(R¹)(R²)(R³)(R⁴)][N⁺(R⁵)(R⁶)(R⁷)(R⁸)]}, each of R¹, R², R³, R⁴, R⁵, R⁶, R⁷ and R⁸ is propyl, and the compound is tetrapropylammoniumtetrathimolybdate. In yet another embodiment where X is {[N⁺(R¹)(R²)(R³)(R⁴)][N⁺(R⁵)(R⁶) (R⁷)(R⁸)]}, each of R¹, R², R³, R⁴, R⁵, R⁶, R⁷ and R⁸ is methyl, and the compound is tetramethylammoniumtetrathimolybdate. In even another embodiment where X is {[N⁺(R¹) (R²)(R³)(R⁴)] [N⁺(R⁵)(R⁶)(R⁷)(R⁸)]}, each of R¹, R², R³, R⁴, R⁵, R⁶, R⁷ and R⁸ is ethyl, and the compound is tetraethylammoniumtetrathimolybdate.

In one embodiment where X is {[N⁺(R¹)(R²)(R³)(R⁴)][N⁺(R⁵)(R⁶) (R⁷)(R⁸)]}, R¹, R² and R³ are independently H, methyl, or ethyl and R⁴ is H or an optionally substituted alkyl, alkenyl, cycloalkyl alkyl, cycloalkyl, aryl, aralkyl, heterocycloalkyl, or heteroaryl. In another embodiment where X is {[N⁺(R¹)(R²)(R³)(R⁴)][N⁺(R⁵)(R⁶)(R⁷) (R⁸)]}, R⁵, R⁶, and R⁷ are independently H, methyl, or ethyl and R⁸ is H or an optionally substituted alkyl, alkenyl, cycloalkyl alkyl, cycloalkyl, aryl, aralkyl, heterocycloalkyl, or heteroaryl. In one embodiment where X is {[N⁺(R¹)(R²)(R³)(R⁴)][N⁺(R⁵)(R⁶)(R⁷)(R⁸)]}, the optional substituents for R⁴ and/or R⁸ are selected from the group consisting of alkyl, OH, NH₂, and oxo. In another embodiment where X is {[N⁺(R¹)(R²)(R³)(R⁴)][N⁺(R⁵)(R⁶)(R⁷)(R⁸)]}, one or more —CH₂— groups of R⁴ and/or R⁸ are replaced with a moiety selected from O, NH, S, S(O), and S(O)₂.

In one embodiment where X is {[N⁺(R¹)(R²)(R³)(R⁴)][N⁺(R⁵)(R⁶) (R⁷)(R⁸)]}, R¹, R², R³, R⁵, R⁶, and R⁷ are independently methyl and R⁴ and R⁸ is each optionally substituted alkyl. In yet another embodiment where X is {[N⁺(R¹)(R²)(R³) (R⁴)][N⁺(R⁵)(R⁶)(R⁷)(R⁸)]}, each of R¹, R², R³, R⁵, R⁶, and R⁷ are independently methyl and R⁴ and R⁸ is each optionally substituted ethyl. In a further embodiment where X is {[N⁺(R¹)(R²)(R³)(R⁴)][N⁺(R⁵)(R⁶)(R⁷)(R⁸)]}, R¹, R², R³, R⁵, R⁶, and R⁷ are independently methyl and R⁴ and R⁸ is each substituted ethyl, wherein the substituent is a hydroxyl. In one embodiment where X is {[N⁺(R¹)(R²)(R³)(R⁴)][N⁺(R⁵)(R⁶)(R⁷)(R⁸)]}, each of R¹, R², R³, R⁵, R⁶, and R⁷ are independently methyl and R⁴ and R⁸ is each —CH₂CH₂—OH.

In one embodiment where X is {[N⁺(R¹)(R²)(R³)(R⁴)][N⁺(R⁵) (R⁶)(R⁷)(R⁸)]}, R¹, R², R³, R⁵, R⁶, and R⁷ are independently methyl; R⁴ and R⁸ is each optionally substituted alkyl; and the compound is tetramethylammoniumtetrathimolybdate. In yet embodiment where X is {[N⁺(R¹)(R²)(R³)(R⁴)][N⁺(R⁵)(R⁶) (R⁷)(R⁸)]}, each of R¹, R², R³, R⁵, R⁶, and R⁷ are independently methyl; R⁴ and R⁸ is each optionally substituted ethyl; and the compound is tetramethylammoniumtetrathimolybdate. In a further embodiment where X is {[N⁺(R¹)(R²)(R³)(R⁴)][N⁺(R⁵)(R⁶)(R⁷) (R⁸)]}, R¹, R², R³, R⁵, R⁶, and R⁷ are independently methyl; R⁴ and R⁸ is each substituted ethyl, wherein the substituent is a hydroxyl; and the compound is tetramethylammoniumtetrathirnolybdate. In one embodiment where X is {[N⁺(R¹)(R²)(R³)(R⁴)][N⁺(R⁵)(R⁶) (R⁷)(R⁸)]}, R¹, R², R³, R⁵, R⁶, and R⁷ are independently methyl; R⁴ and R⁸ is each —CH₂CH₂—OH; and the compound is tetramethylammoniumtetrathimolybdate.

In an exemplary embodiment, the chelator compound is bis-choline tetrathiomolybdate.

In one embodiment, the copper chelator compound according to formula (I) is:

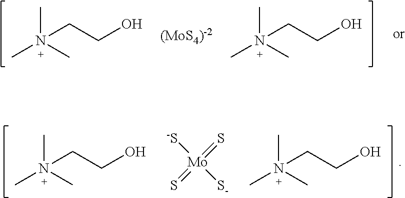

Table 1 provides non-limiting embodiments of where X is {[N⁺(R¹)(R²)(R³)(R⁴)][N⁺(R⁵)(R⁶)(R⁷)(R⁸)]}

TABLE I

| | R¹ | R² | R³ | R⁴ | R⁵ | R⁶ | R⁷ | R⁸ |
|---|---|---|---|---|---|---|---|---|
| 1 | H | H | H | H | H | H | H | H |
| 2 | CH₃ | CH₃ | CH₃ | CH₃ | CH₃ | CH₃ | CH₃ | CH₃ |
| 3 | ethyl | ethyl | ethyl | ethyl | ethyl | ethyl | ethyl | ethyl |
| 4 | propyl | propyl | propyl | propyl | propyl | propyl | propyl | propyl |
| 5 | butyl | butyl | butyl | butyl | butyl | butyl | butyl | butyl |
| 6 | pentyl | pentyl | pentyl | pentyl | pentyl | pentyl | pentyl | pentyl |
| 7 | H | H | H | H | CH₃ | CH₃ | CH₃ | CH₃ |
| 8 | H | H | H | H | ethyl | ethyl | ethyl | ethyl |
| 9 | H | H | H | H | propyl | propyl | propyl | propyl |
| 10 | H | H | H | H | butyl | butyl | butyl | butyl |
| 11 | CH₃ | CH₃ | CH₃ | CH₃ | ethyl | ethyl | ethyl | ethyl |
| 12 | CH₃ | CH₃ | CH₃ | CH₃ | propyl | propyl | propyl | propyl |
| 13 | CH₃ | CH₃ | CH₃ | CH₂CH₂OH | CH₃ | CH₃ | CH₃ | CH₂CH₂OH |

In one embodiment where X is {[N⁺(R¹)(R²)(R³)(R⁴)][N⁺(R⁵)(R⁶) (R⁷)(R⁸)]}, each of [N⁺(R¹)(R²)(R³)(R⁴)] and [N⁺(R⁵) (R⁶)(R⁷)(R⁸)] is independently:

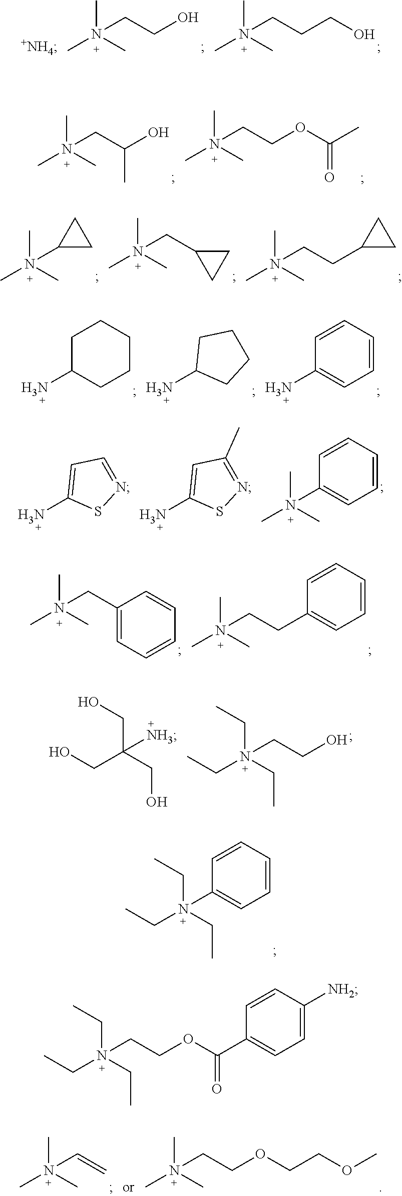

In one embodiment where X is {[N⁺(R¹)(R²)(R³)(R⁴)][N⁺(R⁵)(R⁶) (R⁷)(R⁸)]}, at least one of [N⁺(R¹)(R²)(R³)(R⁴)] and [N⁺(R⁵)(R⁶)(R⁷)(R⁸)] is:

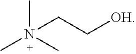

In another embodiment where X is {[N⁺(R¹)(R²)(R³)(R⁴)][N⁺(R⁵) (R⁶)(R⁷)(R⁸)]}, both [N⁺(R¹)(R²)(R³)(R⁴)] and [N⁺(R⁵)(R⁶)(R⁷)(R⁸)] are;

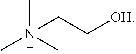

In one embodiment where X is {[N⁺(R¹)(R²)(R³)(R⁴)][N⁺(R⁵)(R⁶) (R⁷)(R⁸)]}, R¹, R², R³ and R⁴ are each independently H or alkyl. In another embodiment, R⁵, R⁶, R⁷ and R⁸ are each independently H or alkyl.

In one embodiment where X is {[N⁺(R¹)(R²)(R³)(R⁴)][N⁺(R⁵)(R⁶) (R⁷)(R⁸)]}, R⁴ and R⁸ are joined by a covalent bond. For example, if R⁴ and R⁸ are both methyl, when R⁴ and R⁸ are joined by a covalent bond, it can form an ethylene link between the two nitrogen atoms as illustrated below:

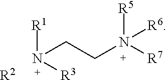

In one embodiment where X is {[N⁺(R¹)(R²)(R³)(R⁴)][N⁺(R⁵)(R⁶) (R⁷)(R⁸)]}, R⁴ and R⁸ are both optionally substituted alkyl group joined by a covalent bond.

In one embodiment where X is {[N⁺(R¹)(R²)(R³)(R⁴)][N⁺(R⁵)(R⁶)(R⁷)(R⁸)]}, R¹, R², R³, R⁵, R⁶, and R⁷ are independently H, methyl, ethyl, or propyl and R⁴ and R⁸ are joined by a covalent bond. In one embodiment, R⁴ and R⁸ is each independently an optionally substituted alkyl group. In one embodiment, the optional substituents for R⁴ and R⁸ is N⁺(R¹⁰)³. In another embodiment, one or more —CH₂— groups of R⁴ and R⁸ are replaced with a moiety selected from the group consisting of O, NH, S, S(O), and S(O)₂.

In one embodiment where X is {[N⁺(R¹)(R²)(R³)(R⁴)][N⁺(R⁵)(R⁶) (R⁷)(R⁸)]}, X is one of:

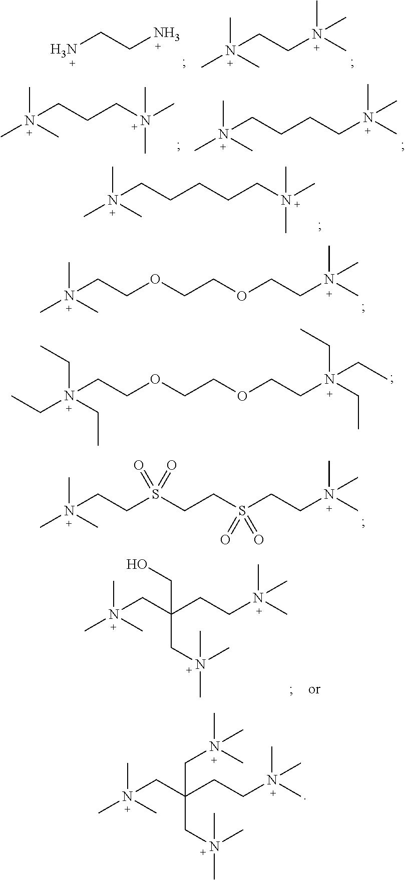

In one embodiment where X is {[N⁺(R¹)(R²)(R³)(R⁴)][N⁺(R⁵)(R⁶) (R⁷)(R⁸)]}, R¹ and R² are each independently H, methyl, or ethyl and R³ and R⁴ are each independently an optionally substituted alkyl, aryl, or aralkyl group. In another embodiment where X is {[N⁺(R¹) (R²)(R³)(R⁴)][N⁺(R⁵)(R⁶)(R⁷)(R⁸)]}, R⁵ and R⁶ are each independently H, methyl, ethyl, or propyl and R⁷ and R⁸ are each independently an optionally substituted alkyl, aryl, or aralkyl group. In one embodiment, the optional substituents for R³, R⁴, R⁷ and R⁸ are OH.

In one embodiment where X is {[N⁺(R¹)(R²)(R³)(R⁴)][N⁺(R⁵)(R⁶) (R⁷)(R⁸)]}, [N⁺(R¹)(R²)(R³)(R⁴)] and/or [N⁺(R⁵)(R⁶) (R⁷)(R⁸)] is independently:

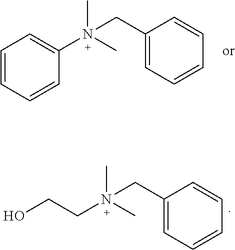

In one embodiment where X is {[N⁺(R¹)(R²)(R³)(R⁴)][N⁺(R⁵)(R⁶) (R⁷)(R⁸)]}, R¹ and R⁴ are each independently H, methyl, ethyl, or propyl and R² and R³ together with N may form an optionally substituted cyclic structure.

In another embodiment where X is {[N⁺(R¹)(R²)(R³)(R⁴)][N⁺(R⁵)(R⁶) (R⁷)(R⁸)]}, R⁵ and R⁸ are each independently H, methyl, ethyl, or propyl, and R⁶ and R⁷ together with N may form an optionally substituted cyclic structure. In one embodiment, one or more —CH₂— groups in R², R³, R⁶ and R⁷ may be replaced with a moiety selected from the group consisting of O, NH, S, S(O), and S(O)₂.

In one embodiment where X is {[N⁺(R¹)(R²)(R³)(R⁴)][N⁺(R⁵)(R⁶) (R⁷)(R⁸)]}, [N⁺(R¹)(R²)(R³)(R⁴)] and/or [N⁺(R⁵)(R⁶) (R⁷)(R⁸)] is independently:

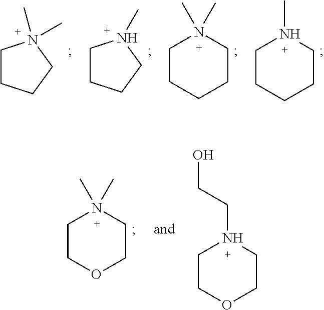

In one embodiment where X is {[N⁺(R¹)(R²)(R³)(R⁴)][N⁺(R⁵)(R⁶) (R⁷)(R⁸)]}, R⁴ and/or R⁸ is absent and R¹ and R² and/or R⁵ and R⁶ together with N forms a optionally substituted 5- or 6-membered aromatic ring, wherein up to 2 carbon atoms in the ring may be replaced with a heteroatom selected from the group consisting of O, N, and S.

In one embodiment where X is {[N⁺(R¹)(R²)(R³)(R⁴)][N⁺(R⁵)(R⁶) (R⁷)(R⁸)]}, [N⁺(R¹)(R²)(R³)(R⁴)] and/or [N⁺(R⁵)(R⁶) (R⁷)(R⁸)]} is independently:

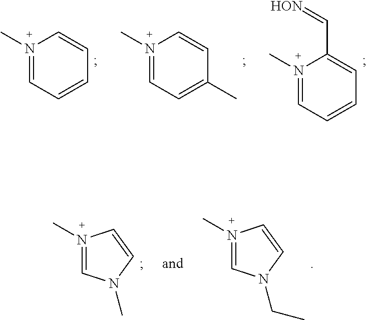

In one embodiment where X is {[N⁺(R¹)(R²)(R³)(R⁴)][N⁺(R⁵) (R⁶)(R⁷)(R⁸)]}, wherein R¹, R², R³, R⁴, R⁵, R⁶, R⁷ and R⁸ are each H.

In an exemplary embodiment, the chelator compound is ammonium tetrathiomolybdate [NH₄]₂MoS₄ (ATTM). ATTM may be combined with other copper chelator compounds, such as ammonium trithiomolybdate [NH₄]₂MoOS₃.

In some exemplary embodiments, COVID-19, a mutation of COVID-19, another Corona Virus with similar mechanisms of action to COVID-19, another virus, or ARDS in a patient is treated by administering a therapeutically effective amount of copper chelator comprising a TTM salt. In one exemplary embodiment, the copper chelator comprises ammonium tetrathiomolybdate [NH₄]₂MoS₄ (or ATTM), and in some exemplary embodiments the copper chelator may further comprise ammonium trithiomolybdate [NH₄]₂MoOS₃. The amount of a TTM salt delivered is individualized. In an exemplary embodiment, the therapeutically effective amount of the copper chelator delivers between 90 and 180 mg of TTM/day. The amount of TTM is adjusted according to the level of the ceruloplasmin in plasma. The effective copper chelation is achieved when the plasma ceruloplasmin level approaches 50% of the normal level; i.e. 15-17 mg/dl.

The copper chelator may be administered in a composition comprising pharmaceutically acceptable carriers and/or excipients. The compositions may be administered in an intravenous form or an oral form, such as a tablet, a microtablet, or a capsule. In some exemplary embodiments, the copper chelator may be in composition of an oral form with specific carriers, coatings and/or excipients that provide a delayed release of the copper chelator after passage through the stomach. Specifically, the carriers and/or excipients are selected to facilitate protection of the copper chelator against destruction by gastric acid and enabling optimal intestinal uptake and absorption. For example, the oral forms of the composition may include an enteric coating of the tablet or capsule or include a delayed release preparation.

In some exemplary embodiments, the patient is treated by administering a therapeutically effective amount of copper chelator comprising a TTM salt in concert with another antiviral composition, antibody, or another treatment for a Corona virus, such as COVID-19, mutation of COVID-19, or another Corona Virus with similar mechanisms of action to COVID-19 or Influenza.

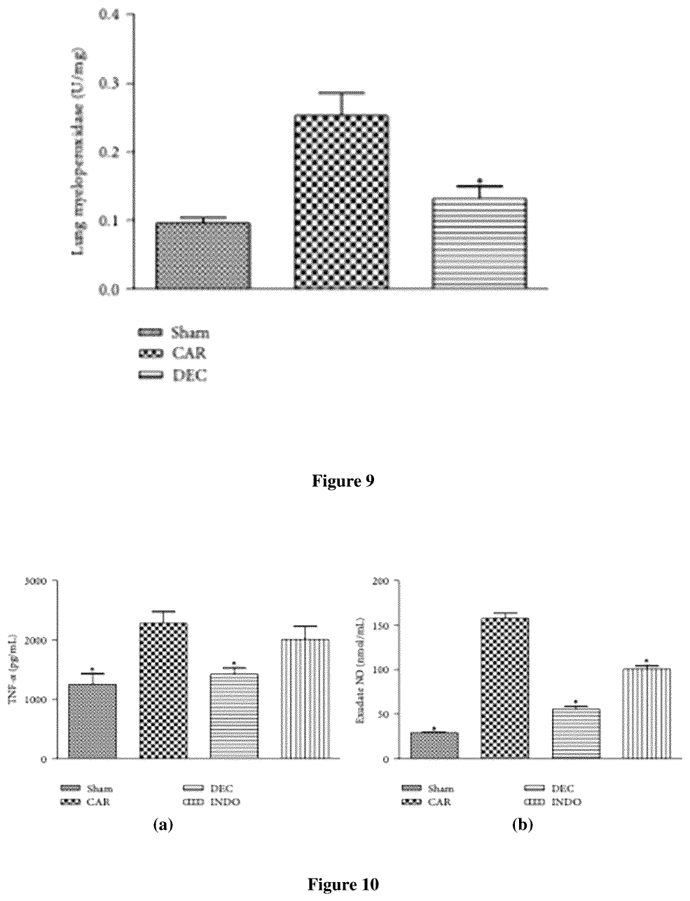

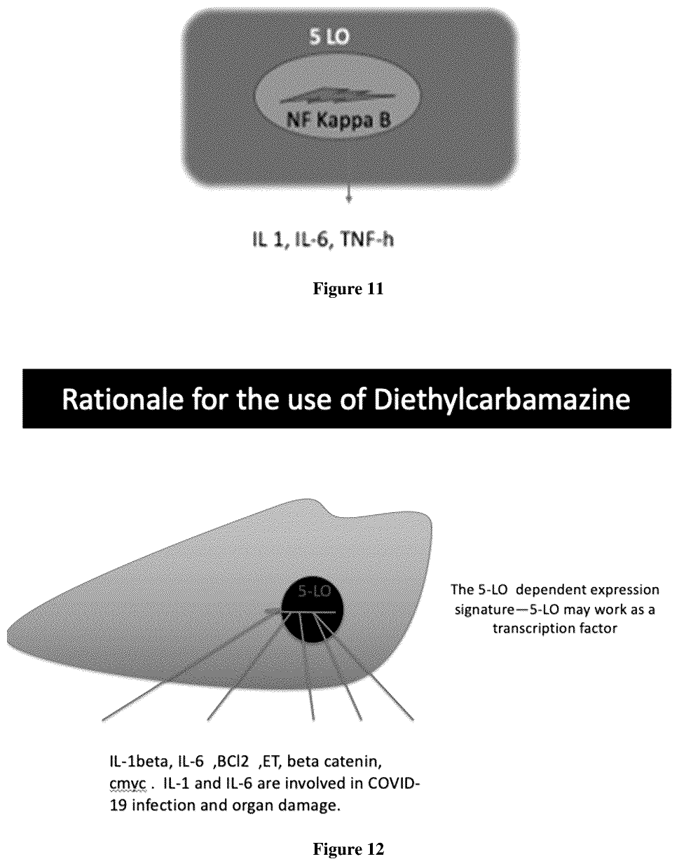

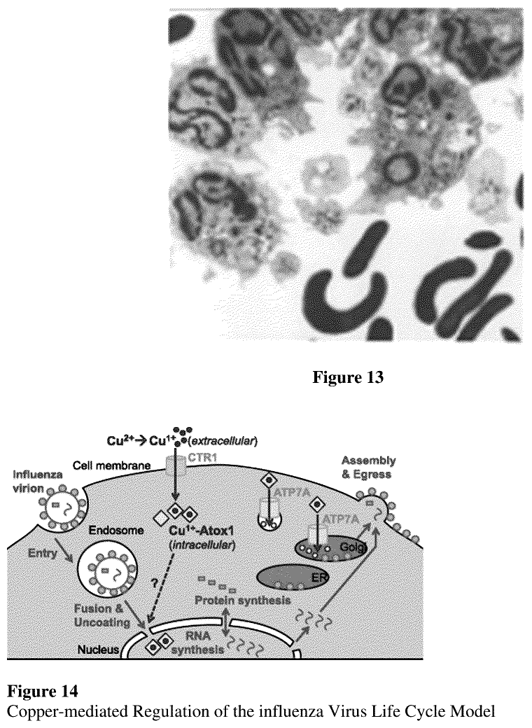

In other exemplary embodiments, in addition to TTM, a 5-lipoxygenase inhibitor (DEC or Zileuton) is to be used in concert with TTM for the disease prevention treatment strategy for COVID-19, mutation of COVID-19, or another Corona Virus with similar mechanisms of action to COVID-19 or Influenza. DEC or Zileuton are-inhibitors of the 5-lipoxygenase enzyme (5-LO), which drives inflammation and likely contributes to endothelial-damage and lung injury (FIGS. 9 and 10 (a) and (b)).

Thus, it is likely that the copper chelator comprising a TTM salt, and a 5-LO inhibitor work synergistically in preventing intravascular inflammation. While a TTM salt is expected to reduce vascular permeability and chemotaxis, inhibition of 5-LO is expected to decrease inflammation, inhibit neutrophil chemotaxis and NF-kappaB-dependent gene transcription. The 5-LO enzyme that is expressed in activated lung vessel endothelial cells acts in the context of pulmonary vascular disease as an activator of gene expression. 5-LO leads to the production of leukotriene C4, which is the first and well-established action of 5-LO, and leukotriene C4 increases pulmonary vasoconstriction by contracting smooth muscle cells in the bronchial airways and in the lung vessels. Thus, inhibiting 5-LO would also inhibit leukotriene C4 synthesis, which would remove a pulmonary vessel constricting substance. A second action is a non-enzymatic function of binding to the 5-LO activating protein (FLAP) on the envelope of the cell nucleus. Fitzpatrick and Lepley showed in 1998 that 5-LO co-precipitated with a subunit of the transcription factor NF-kappaB when they examined nuclear extracts. NF-kappaB controls the expression of genes encoding several LTB4 inflammatory mediators. Thus, 5-LO, by binding to NF-kappaB in the cell nucleus could activate transcription of a number of genes in control of cell growth and genes encoding inflammatory mediators such as IL-1beta and IL-6-and also VEGF. As a result of 5-LO inhibitor treatment, there could be a reduction in the vascular inflammation and perhaps stem cell reprogramming leading to halting of disease progression and assist disease reversal. LTB4 is another important chemotactic leukotriene that is a product of the enzyme leukotriene A4 hydrolase—which is downstream from 5-LO. LTB4-has recently been studied in rats and it was demonstrated that LTB4 caused pulmonary-endothelial cell apoptosis (Tian W. et al Sci Transl Med. 2013 Aug. 28;5(200):200ra117). Because effective inhibition of the 5-LO would also block LTB4 production, it is expected that 5-LO inhibitors in the treatment of COVID-19 induced diseases and Influenza would also target LTB4-dependent pathomechanisms.

In some other exemplary embodiments, a patient suffering from a Corona Virus, such as COVID-19, a mutation of COVID-19, or another Corona Viruses with similar mechanisms of action to COVID-19, or ARDS or influenza may be treated by the administration of a therapeutically effective amount of a 5-LO inhibitor, such as DEC, in concert with another antiviral composition or another Corona Virus treatment.

In some exemplary embodiments, COVID-19, mutation of COVID-19, another Corona Virus with similar mechanisms of action to COVID-19, or ARDS or influenza in a patient may be treated with a therapeutically effective amount of a combination of copper chelator comprising a TTM salt and at least one 5-LO inhibitor. In particular, treatment of patients with severe forms of COVID-19 induced organ damage, and treatment in order to prevent development of organ damage, is possible with the administration of a therapeutically effective number of one of the 5-LO inhibitors Diethylcarbamazine or Zileuton in combination with a therapeutically effective amount of a copper chelator comprising a TTM salt.

In some exemplary embodiments, COVID-19, mutation of COVID-19, an another Corona Virus with similar mechanisms of action to COVID-19, or ARDS or influenza in a patient may be treated with a therapeutically effective amount of a combination of copper chelator comprising a TTM salt and at least one 5-LO inhibitor and in particular, treatment of patients with severe forms of COVID-19 induced organ damage, and treatment in order to prevent development of organ damage, is possible with the administration of a therapeutically effective number of one of the 5-LO inhibitors Diethylcarbamazine or Zileuton in combination with a therapeutically effective amount of a copper chelator comprising a TTM salt.

Fluvoxamine is an approved anti-depressant and anti-anxiety agent that inhibits Inositol Requiring Enzyme 1 (IRE 1) dependent production of inflammatory signals—in particular those following the activation of Toll-like receptors (TLR). Knock out of the gene encoding the Sigma-1 receptor in cells showed an increased expression of IL-6—which persisted after an inhibitor of NF-kappaB had been added—suggesting that the anti-inflammatory action of

Fluvoxamine was independent of NF-kappaB.

Sulforaphane activates the transcription factor Nrf2. This transcription factor is a switchboard that transcribes a host of antioxidant enzyme genes-resulting in the production of antioxidant enzymes, such as superoxide dismutase and catalase. Because inflammation is associated with oxidant stress, sulforaphane reduces the oxidant stress component of inflammation and is a suitable co-drug.

Apigenin also affects, or specifically inhibits, NF-kappaB, and, thus, Apigenin is also a suitable co-drug. Such an NF-kappaB inhibitor could be considered for the treatment of incident severe PAH—either alone or in combination with either a copper chelator comprising a TTM salt or the 5-LO inhibitors. Indeed, one inhibitor of NF-kappaB, pyrrolidine dithiocarbamate, has been shown to reverse established angio-obliterative PAH in the preclinical rat model of Sugen/hypoxia-induced PAH (Farkas D. et al AJRCMB, Vol. 15, No. 3, Sep. 1, 2014). These data illustrate that NF-kappaB plays a role in pulmonary vascular remodeling, and one can predict inhibition of NF-kappaB in the setting of COVID-19-induced intravascular inflammation or Influenza inhibits the production of inflammatory mediators like IL-1 and _L-6 and TNF-alpha. Such an action could prevent the progression from COVID-19 infection to organ damage. Apigenin, in particular, has been shown to reduce inflammation. Thus, because 5-LO and the transcription factor NF-kappaB interact in the cell nucleus to initiate the expression of inflammatory and cell growth-promoting genes, then there would be an expected synergism in the drug action of a 5-LO inhibitor, such as Zileuton or DEC, and Apigenin. Likewise, the combination of Apigenin with a copper chelator comprising a TTM salt is expected to provide the combined anti-inflammatory action of Apigenin and the anti-angiogenic, anoikis-inducing action of the copper chelator comprising a TTM salt. Thus, some exemplary embodiments of concern are treating a patient suffering from PAH by administering a therapeutically effective amount of Apigenin with a therapeutically effective amount of the copper chelator comprising a TTM salt.

Indole-3-carbinol (i3c) is another NF-kappaB inhibitor, and, thus, considered to be a suitable co-drug. I3c is a plant derived compound with a pleiotropic action profile, and it has been demonstrated that i3c has anti-inflammatory and anti-tumor growth activities. Specifically, i3c intervenes in signal transduction and controls cell growth by affecting several receptors and transcription factors. It inhibits the inflammation switchboard NF-kappaB and also is a ligand for the aryl hydrocarbon receptor (AhR), which is involved in drug metabolism and has been recently targeted for cancer therapy. Recently, it has been shown that i3c can upregulate the activity of the important tumor suppressor PTEN. Each of these pathways can explain the anti-inflammatory and anti-tumor effects of i3c. Levels of PTEN have been shown to be reduced in the lungs from pulmonary hypertensive animals and several experimental studies have shown that pulmonary vascular remodeling can be modulated in a PTEN-dependent manner. Thus, because inflammation and uncontrolled vascular cell growth are hallmarks of both PAH and cancer. Treatment of PAH patients with i3c alone, or in combination with a copper chelator comprising a TTM salt, may reverse the pulmonary vascular lumen obliteration by inhibiting abnormal cell growth and inhibiting inflammation. Such a mechanism of action may be useful in preventing the progression from intravascular inflammation to COVID-19-triggered organ damage. Some exemplary embodiments of the present invention comprise treating a patient suffering from COVID-19 infection and susceptible to organ damage by administering a therapeutically effective amount of i3c with a therapeutically effective amount of a copper chelator comprising a TTM salt.

Bufalin is another co-drug for consideration that may also have anti-inflammatory actions via inhibition of NF-kappaB and inhibition of the expression of the matrix metalloproteinases MMP2 and MMP9. Bufalin can also reduce the expression of the integrin alpha2/beta5. Significantly, Bufalin is a multi-target anti-cancer agent, which appears to be promising for cancer treatment, and in several studies Bufalin has been shown to inhibit the epithelial mesenchymal transition (EMT) in cancers-one of the hallmarks of cancer. This EMT inhibition occurs by downregulation of TGF beta receptor expression in lung cancer cells. This is considered relevant to PAH treatment because in PAH there is endothelial mesenchymal transition (EnMT) which is likewise TGF beta signaling dependent, Bufalin is expected to inhibit EnMT in the sick lung vessels. In particular, the “plugs” occluding the vessel lumen in angioproliferative PAH consists of phenotypically altered cells (some have undergone EnMT), and very likely these cells rest on abnormal matrix proteins and these cells also very likely have undergone integrin switching. It is possible that a compound like Bufalin may dissolve the cellular plug by interrupting TGF beta signaling and induce anoikis by altering abnormal integrins. Bufalin has not yet been clinically tested. However, Bufalin's multi-modal action profile makes it a candidate as a co-drug with a copper chelator comprising a TTM salt in COVID triggered diseases and influenza; and some exemplary embodiments of the present invention comprise treating a patient suffering from COVID-triggered disease and by administering a therapeutically effective amount of Bufalin with a therapeutically effective amount of a copper chelator comprising a TTM salt.

Other possible co-drugs include naturally occurring plant products, specifically Baicalin, Curcumin, and Quercetin, which are useful for the treatment of inflammatory disorders. These compounds were identified as copper handling modifiers in a Chinese publication that analyzed studies where plant extracts in various combinations were used to treat patients with the copper storage Wilson disease (Xu M-B, Rong P-Q et al, Front in Pharmacol, 2019). The authors reference experimental data indicating that Curcumin, Baicalin and Quercetin can alter intracellular copper handling. However, each of these compounds have been shown to possess other activities which are also relevant to treating COVID-19 and influenza related inflammation and organ damage.

Of these three compounds, Baicalin has received the most attention in recent years. Baicalin is an extract from a Chinese herb that has been used to treat many diseases in China for centuries. In particular, there are three findings that are most relevant to treating intravascular inflammation. First, a high dosage of Baicalin was found to inhibit angiogenesis. This relevant because in ARDS there is pulmonary thrombosis due to endothelial cell damage and, thus, Baicalin could inhibit intravascular inflammation and thrombosis. Second Baicalin was found to alleviate silica-induced lung inflammation and fibrosis by inhibiting T-helper 17 cell (TH 17), or more broadly it was shown to stimulate Tregs and be an anti-inflammatory. This is relevant because Baicalin could, thus, inhibit pulmonary vascular inflammatory cell infiltration. Third, Baicalin was found to attenuate monocrotaline-induced pulmonary hypertension through the bone morphogenetic protein signaling pathway, having an anti-inflammatory effect. This is relevant because of the beneficial effect in this model of inflammation-triggered endothelial cell damage.

Recently published results obtained in rat models of pulmonary arterial hypertension (PAH) demonstrate that pharmacological inhibition of HIF-2α may be a promising novel therapeutic strategy for the treatment of severe vascular remodeling and right heart failure in patients with PAH. Both pulmonary hypertension and right heart failure have been described in COVID-19 infected patients. Thus, both a copper chelator comprising a TTM salt and Baicalin inhibit HIF-1α, and a copper chelator comprising a TTM salt also inhibits HIF-2α.

It is possible that adding Baicalin to a dose of a copper chelator comprising a TTM salt may prevent endothelial cell death and intravascular inflammation. Thus, in some exemplary embodiments a patient suffering from an infection caused by COVID-19, a mutation of COVID-19, another Corona Virus with similar mechanisms of action to COVID-19, or at risk of developing ARDS, or influenza can be treated by administering a therapeutically effective amount of the copper chelator comprising a TTM salt and a therapeutically effective amount of Baicalin.

Curcumin is a diarylheptanoid extracted from turmeric, which has long been considered to have anticancer effects, and there is a voluminous literature describing the effects of Curcumin in many models of cancer and inflammatory diseases. However, while the literature focuses on its antioxidant and anti-inflammatory properties, Curcumin has also been shown to induce the pulmonary anti-hypertensive Heme oxygenase 1 and has protective effects against lung injury via TGF beta 1 inhibition. It suppresses gastric carcinoma by inducing apoptosis of the tumor cells. Thus, Curcumin may inhibit the inflammatory component of pulmonary vascular remodeling, and it is possible that Curcumin could inhibit pulmonary vascular cell proliferation via inhibition of the signal transducer and activator of transcription 3 (STAT3) signaling pathway. Because inflammation is an important component of the angiopathy in severe PAH, Curcumin may be a non-toxic partner with a copper chelator comprising a TTM salt in the treatment or prevention of severe COVID-19-induced disease. Indeed, it has been shown that Curcumin derivatives were mild phosphodiesterase V inhibitors (acting like a pulmonary vasodilator), and, it has been suggested that Curcumin could be used to treat PAH. Thus, in some exemplary embodiments a patient suffering from COVID-19, a mutation of COVID-19, or another Corona Virus with similar mechanisms of action to COVID-19-induced infection and/or at risk of developing ARDS, or influenza can be treated by administering a therapeutically effective amount of the copper chelator comprising a TTM salt and a therapeutically effective amount of Curcumin.

Quercetin is a plant flavonoid contained in many plants and vegetables like broccoli and onions, and its antioxidant activities are well-documented. However, Quercetin has also been shown to inhibit VEGF expression and VEGF receptor 2 signaling, and, thus, it is anti-angiogenic. It has also been shown to inhibit glycolysis in breast cancer cells (one of the hallmarks of cancer), vascular remodeling in rodent models of PH, and endothelial-mesenchymal transformation (EnMT). Moreover, it has been shown that Quercetin improves wound healing by modifying the integrin alpha v/beta 1. Thus, because of its antioxidant action profile, Quercetin may inhibit the intravascular disease component of Covid 19-induced diseases. Thus, in some exemplary embodiments a patient suffering from severe COVID-19 a mutation of COVID-19, or another Corona Virus with similar mechanisms of action to COVID-19-induced disease or at risk of developing ARDS or influenza can be treated by administering a therapeutically effective amount of the copper chelator comprising a TTM salt and a therapeutically effective amount of Quercetin.

Applied Therapeutics Aldose Reductase inhibitor AT-001 is designed for Diabetic Cardiomyopathy, it has antioxidant properties and may work in tandem with the two drugs (5-lipoxygenase inhibitor+TTM) to mitigate acute lung inflammation.

Beraprost is a stable Prostacyclin Analogue which has been used in an oral form for the treatment of severe forms of Pulmonary Arterial Hypertension. Beraprost is a vasodilator and has additional anti-inflammatory and antifibrotic activities. Beraprost protects endothelial cells and may be effective is strengthening the endothelial cells for treating ARDS.

The known and expected effects of the copper chelator comprising a TTM salt (listed as “TTM”) and the above discussed other active agents, or co-drugs are summarized below in Table 2.

TABLE 2

| | Expected Effect On COVID | Expected Combined |
|---|---|---|
| | 19, Covid 19 mutations and | Effect with |
| Drug agent | variants, ARDS, and Influenza. | TTM |
| TTM | Anti-angiogenesis | |
| | Anti-inflammation | |
| | Modification of stem cell | |
| | behavior | |
| 5-LO inhibitors, e.g. DEC and | Anti-inflammation | TTM & 5-LO inhibitions are |
| Zileuton. | Inhibition of pulmonary | synergistic: TTM is anti-angiogenic |
| | vasoconstriction | and 5-LO inhibition decreases |
| | | inflammation. |
| Baicalin | Anti-inflammation | TTM & Baicalin are both inducing |
| | | anoikis-but by different molecular |
| | | mechanisms. |
| Inhibitors of NF-kappaB, such | Inhibition of NF-kappaB | TTM is anti-angiogenic and NF- |
| as Apigenin | dependent gene | kappaB inhibitors are anti- |
| | transcription | inflammatory |
| Bufalin | Reopening of occluded lung | Induction of death of abnormal |
| | vessels | cells, prevention of intravascular |
| | | plugs, likely via inhibition of TGF |
| | | beta signaling, synergistic with |
| | | TTM |
| Quercetin | Decrease in inflammatory | Anti-inflammation synergism with |
| | cells in the lung vascular | TTM. |
| | lesions | |
| Curcumin | Decrease in the | Anti-inflammation synergism with |
| | inflammatory cells in the | TTM. |
| | lung vascular lesions and | |
| | pulmonary vasodilation. | |
| | Effect on copper handling | |
| | in cell | |
| Applied Therapeutics Aldose | Antioxidant action | Potentially synergistic with TTM |
| Reductase inhibitor AT-001 | | |
| Fluvoxamine (SSRIs) | Anti-inflammation | Anti-inflammation synergism with |
| | | TTM, Beraprost and DEC |
| Beraprost | A stable Prostacyclin | Synergistic with DEC |
| | Analogue, vasodilator, | |
| | anti-inflammatory and | |
| | antifibrotic-Protects | |
| | endothelial cells | |
| Sulforaphane | Anti-inflammation | Synergistic with DEC and |
| | | TTM |
| Prostacyclin Analogues, such as | Anti-inflammatory, | Anti-inflammatory synergism on |
| Iloprost and the PGI₂ Receptor | antifibrotic, vasodilator, | the level of cell-cell interactions, |
| Agonist Selexipag, Treprostinil | Protecting the endothelial | chemotaxis, protecting against |
| and Beraprost. | cell barrier, anti-thrombotic | clotting, protecting against vascular |
| | | leak |

In terms of administration, some exemplary embodiments concerning the administration of both the copper chelator comprising the TTM salt and one or more co-drugs in a single dose form or composition. In other exemplary embodiments the copper chelator comprising a TTM salt, and co-drugs are administered in separate compositions, which may be administered via the same route. Alternatively, these separate compositions may be administered by different routes. For example, the copper chelator comprising a TTM salt may be in an oral form, or an intravenous form and the co-drug may be in composition of an oral, intravenous, or inhalable form.

In some exemplary embodiments, the TTM salt—with or without a co-drug—is administered 90 to 180 mg/day, which is adjusted to the target ceruloplasmin level of 50% of its normal value; for practical purposes this target is 15-17 mg/dl of plasma.

The compositions may comprise pharmaceutically acceptable carriers and/or excipients. The compositions may be in an intravenous form or an oral form, such as a tablet, a microtablet, or a capsule. For compositions comprising the copper chelator comprising a TTM salt, with or without the co-drugs, specific carriers and/or excipients may be added to provide a delayed release of the TTM salt after passage through the stomach. Specifically, the carriers and/or excipients are selected to facilitate (1) protection of the TTM salt against destruction by gastric acid and enabling optimal intestinal uptake and absorption and (2) any interaction with co-drugs that may be combined in the same pill. For example, composition in an oral form may include an enteric coating of the tablet or capsule or include a delayed release preparation. Such a composition may release, for example: (1) a TTM salt after the oral form of TTM passes the stomach and (2) at least one other active agent released in the stomach or after the other active agent passes the stomach. Alternatively, if the composition does not include an enteric coating, it is possible delay release of the TTM salt after passage through the stomach by co-administering a therapeutically effective amount of a proton pump inhibitor. The proton pump inhibitor may be included in the same oral form as the TTM salt or in a separately administered composition.

In some embodiments, a single dose may administer the copper chelator comprising a TTM salt, at least one of DEC or Zileuton, and, optionally, the proton pump inhibitor. As explained above, the copper chelator comprising a TTM salt and one or more co-drugs may be administered in a single dose form or composition or in separate compositions. Also, as explained above, these separate compositions may be administered by different routes, such as the copper chelator comprising a TTM salt being in an oral form or an intravenous form with the co-drug(s) being in composition of an oral, intravenous, or inhalable form. Thus, a single dose of the copper chelator comprising a TTM salt, at least one of DEC or Zileuton, and, optionally, a proton pump inhibitor, may be administered in a variety of forms for administration by different routes, such as:
(a) oral administration comprising a combination oral form of the copper chelator comprising a TTM salt and DEC or Zileuton, all of which are within either an enteric coated capsule or a tablet; for the enteric coated capsule, the copper chelator comprising a TTM salt is separated from the DEC or Zileuton by a coating or sealed in in a separate compartment of the capsule or from the DEC or Zileuton, and for the tablet, TTM and DEC or Zileuton are separated by a barrier or coating.
(b) oral administration comprising a first oral form being a combination oral form of the copper chelator comprising a TTM salt with DEC or Zileuton, the copper chelator comprising a TTM salt being separately sealed or otherwise isolated from DEC or Zileuton, the first oral form being a capsule without an enteric coating, and a second oral form of the required proton pump inhibitor, such proton pump inhibitor protecting the TTM salt from stomach acid.
(c) oral administration comprising three separate oral forms, optionally packaged together: a first oral form comprising the copper chelator comprising a TTM salt without an enteric coating, a second oral form comprising DEC or Zileuton, and a third oral form comprising a proton pump inhibitor.
(d) oral administration comprising a first oral form including the copper chelator comprising a TTM salt, without enteric coating, and a second oral from, which includes the combination of DEC or Zileuton and a proton pump inhibitor.
(e) oral administration comprising a first oral form including a combination of the copper chelator comprising a TTM salt and a proton pump inhibitor, each sealed from each other, no enteric coating, and a second oral form comprising DEC or Zileuton.
(f) a combination of administration routes, wherein the copper chelator comprising a TTM salt is administered in an oral form, either with and without enteric coating, and DEC or Zileuton is administered in an inhaled or intravenous form.
(g) a combination of administration routes, wherein the copper chelator comprising a TTM salt is administered as in intravenous form and DEC or Zileuton is administered in an oral, inhaled, or intravenous form.
(h) a combination of administration routes, wherein the copper chelator comprising a TTM salt is administered in an intravenous form and DEC or Zileuton is administered in an inhaled form through an inhaler with at least one of Beraprost or Fluvoxamine, e.g., the inhaled form may comprise DEC with Beraprost, a combination of DEC with Beraprost and Fluvoxamine, a combination of Zileuton with Beraprost, or a combination of Zileuton with Beraprost and Fluvoxamine or a combination of the foregoing where sulforaphane is included with the inhaled Beraprost with or without DEC or Zileuton.
(i) a combination of administration routes, wherein the copper chelator comprising a TTM salt is administered in an intravenous form and DEC or Zileuton is administered in an inhaled form comprising Fluvoxamine through an inhaler.

The inventive compositions of TTM and DEC may be administered in a variety of oral delivery dosage forms including, but not limited to tablets comprising active coatings (e.g., enteric coatings), hard shell capsules containing a combination of pellets with and without enteric coatings pellets, multilayer enterically-coated tablets, normal multicomponent tablets (which may be also enterically coated), enterically coated capsules, small enterically coated capsules that release each active in a different location, minitablets, granulations, hard capsules including enteric capsules combined with powders, granules, pellets, and/or minitablets, etc., and all of the these delivery dosage forms may be filled into hard capsules. Further forms may include a hard-shell capsule or an enteric capsule that includes melt extruded TTM and DEC conventionally processed or melt extruded, or a dosage form produced by 3D printing technology.

For example, in one exemplary embodiment, the dosage form may utilize active coating (DEC in coating) on top of an enteric coating of a tablet containing TTM. In this embodiment a tablet of TTM is made with respective ingredients and enterically coated. Then an active coating step (i.e., DEC is contained in a soluble spray solution or as a powder or by tablet-in-tablet compression) is applied to the tablets. As a result, DEC is released immediately and the TTM is protected from acid stomach content. Also in this embodiment, a topcoat may be added to provide the active coat from falling apart.

In another exemplary embodiment, the dosage form may be MUPS (multi-unit pellet system): compress enterically-coated pellets with TTM in a matrix containing DEC. In this embodiment, TTM is processed to micro pellets with a diameter below that of tablets, each pellet is enterically coated, the enteric coating isolates TTM from DEC or other co-drugs that may be added. The TTM micro pellets are then compressed with a mixture of suitable fillers, disintegrants and other excipients, also containing DEC (or other co-drugs) in one pill. Such tablets may be coated with an appearance coat.

In another exemplary embodiment, the dosage form may be a hard-shell capsule containing enterically coated pellets of TTM and pellets/powder of DEC. In this embodiment, TTM is processed into micro-pellets that are enterically coated, and then filled in a hard-shell capsule together with DEC. In this embodiment, a binder may be added to allow the DEC to bind together in a hard pill, where there is no enteric coating for the DEC, and the TTM is contained within the DEC or within DEC and the binder created pill.

In another exemplary embodiment, the dosage form may be a multilayer tablet enterically coated (i.e., both drugs are released in small intestine). In one embodiment, a three-layer pill is manufactured whereby the DEC is on one layer, the middle layer isolates the DEC and the TTM, and the TTM is contained in the third layer. The tablets are enterically coated as a total to protect TTM. The TTM in this embodiment is enterically in this layer or the TTM in this layer are TTM microcapsules that are enterically coated and manufactured into a layer with a binder that may or may not be an enteric coating. Such tablets may be coated with an appearance coat.

In another exemplary embodiment, the dosage form may be a normal multicomponent tablet (formulated with both TTM & DEC) also enterically coated (i.e., both drugs are released in small intestine). In this embodiment both TTM and DEC are formulated with suitable tableting excipients to form a single layer compressed tablet which is then enterically coated to protect TTM. Possibly either TTM or DEC is coated to avoid any chemical reactions leading to unstable formulations.

In another exemplary embodiment, the dosage form may be an enterically coated capsule containing TTM and DEC (i.e., both drugs are released in small intestine). In this embodiment, both the TTM and the DEC are filled in suitable form (powder, micro-tablets, pellets, granulate, beads) in an enterically coated capsule. Possibly either TTM or DEC is coated to avoid any chemical reactions leading to unstable formulations.

In another exemplary embodiment, the dosage form may be a small enterically coated capsule containing TTM inside a capsule with DEC (DEC is released in the stomach and TTM is releases in the small intestine) In this embodiment, the TTM is filled into a smaller enterically coated capsule and this TTM filled capsule, together with DEC, is used to fill and larger conventional capsule. A conventional capsule is a capsule that disintegrates rapidly in acidic dissolution media made from excipients like gelatin or HPMC. In this embodiment the larger capsule will dissolve in the stomach, releasing the DEC, and the smaller enterically coated TTM capsule will pass through the stomach and releasing the TTM in the small intestine.

In another exemplary embodiment, the dosage form may be a TTM filled into an enteric capsule which is then placed into a hard-shell capsule along with DEC powder, granules, pellets, minitablets etc. In this embodiment the hard-shell capsule will dissolve in the stomach, releasing the DEC, and the smaller enterically coated TTM capsule will pass through the stomach and releasing the TTM in the small intestine. Hard Shell capsules made from HPMC and HPMCAS (HPMC acetate succinate) or other enteric polymers which offers resistance to disintegration in acidic media.

In another exemplary embodiment, the dosage may be in the form of minitablets of TTM and DEC in combination or individually made by granulating or blending with suitable excipients and compressing these minitablets on a rotary tablet press. Such minitablets may then be coated with enteric polymers. These minitablets are then filled into a hard-shell capsule. In this embodiment the TTM may be coated with an enteric polymer and the DEC with or without an enteric Polymer.

In another exemplary embodiment, the dosage form may be TTM and/or DEC granulated with suitable enteric polymers. Such granulations are then compressed into a monolithic or multilayer tablet. Presentations could include; monolithic or multilayer tablets containing enteric granulation of TTM and non-enteric granulation of DEC and possible combinations (i.e., enteric TTM+enteric DEC, non-enteric TTM, enteric DEC, non-enteric TTM+non-enteric DEC). In another embodiment, layers of TTM and DEC granulations may be separated with an inert layer. Such tablets can be coated with an appearance coat.

Any of the capsule presentations above filled into a hard-shell capsule which is then coated with enteric polymers.

In another exemplary embodiment for a dosage form, TTM can be melt extruded together with enteric polymers and the extrudates can then be either filled along with similarly processed DEC or conventionally processed DEC in a hard-shell capsule or into an enteric capsule. Alternatively, such extrudates can also be molded into a single tablet using melt extrusion techniques. In this embodiment, the tablet dissolves in the stomach first, releasing the DEC, and then the TTM that has been formulated with an extruded enteric polymer, passes from the stomach to release in the small intestine.

In another exemplary embodiment for a dosage form, the desired release profile and separation between DEC and TTM can also be achieved by various 3D printing technologies currently under development.

In other exemplary embodiments a two-part pill comprising of a small enteric coated capsule containing 15 to 100 mg of TTM with or without a bulking agent, such bulking agent to assure the TTM capsule does not collapse when the volume of TTM is not enough to fill the capsule, and such capsule is inserted into a larger capsule that also is filled with DEC and will dissolve in the stomach. The DEC in the outer pill will comprise a range from 50 mg to 350 mg of DEC.

In other exemplary embodiments, any of the above-described exemplary embodiments of dosage forms of TTM and DEC or any of the oral forms may also be taken with one or more of the following as part of a single dose, as an additional oral form or as an additional component to one of the oral forms: Selective Serotonin Reuptake Inhibitors (SSRIs), such as Fluvoxamine, Sulforaphane, Apigenin, Indole-3-carbinol (i3c), Bufalin, the Applied Therapeutics Aldose Reductase inhibitor AT-001, Baicalin, Curcumin and Quercetin. Alternatively, with respect to Baicalin, Curcumin and Quercetin, in an Intensive Care Unit setting, these agents can be administered as a slurry via a gastro-intestinal tube in order to achieve a higher bioavailability.

In another exemplary embodiment, a single dose may administer the copper chelator comprising a TTM salt, at least one of DEC or Zileuton, with or without Ivermectin, and, optionally, the proton pump inhibitor. Such a single dose may include, for example, a first oral firm comprising DEC or Zileuton plus Ivermectin and a second oral form comprising the copper chelator comprising a TTM salt with an enteric coating. This single dose may also be taken with one or more of the following as an additional oral form or in combination with the first or second oral form: Fluvoxamine, Sulforaphane, Apigenin, Indole-3-carbinol (i3c), Bufalin, the Applied Therapeutics Aldose Reductase inhibitor AT-001, Baicalin, Curcumin and Quercetin. Alternatively, with respect to Baicalin, Curcumin and Quercetin, in an Intensive Care Unit setting, these agents can be administered as a slurry via a gastro-intestinal tube in order to achieve a higher bioavailability.

In other exemplary embodiments, any of the foregoing combinations of TTM and DEC, TTM and Zileuton, with other active agents, for administration by a variety of forms via different routes, with or without Ivermectin, Fluvoxamine, Sulforaphane, Apigenin, Indole-3-carbinol (i3c), Bufalin, the Applied Therapeutics Aldose Reductase inhibitor AT-001, Baicalin, Curcumin and Quercetin, may also be administered in concert with current and potential drugs that target a corona virus, such as COVID-19, via different mechanisms of action, such as AT-527 (Atea Pharmceutical's antiviral therapeutic), silmitasertib (Taiwan-based Senhwa Biosciences), Gil remdesivir (Gilead Science Inc.), ilteritinib (made by Japan-based Astellas Pharma), abemaciclib (Eli Lily) and ralimetinib, dasatinib (Bristol-Meyers Squibb) and as Favipiravir (or “FABIFLU” in India) and Avifavir (in Russia), which is associated with Favipiravir and developed by Fujifilm Toyama Chemical of Japan and Pfizer's antiviral agent named PF-07321332, a protease inhibitor that provided anti-viral activity against Covid 19.

In other exemplary embodiments, any of the foregoing combinations of TTM and DEC, TTM and Zileuton, with other active agents, for administration by a variety of forms via different routes, with or without Ivermectin, Selective Serotonin Reuptake Inhibitors (SSRIs), such as Fluvoxamine, Sulforaphane, Apigenin, Indole-3-carbinol (i3c), Bufalin, the Applied Therapeutics Aldose Reductase inhibitor AT-001, Baicalin, Curcumin and Quercetin, may also be administered in concert with drugs that contain the corona virus or in the case of a different virus, anti-bodies and any other antiviral drugs.

In an alternative embodiment, treatment of Corona Virus, such as is COVID-19, a mutation of COVID-19, another Corona Virus with similar mechanisms of action to COVID-19, or ARDS, or Influenza in a patient in need thereof may be carried out by administering to the patient a therapeutically effective amount of Diethylcarbamazine (DEC) or Zileuton with a therapeutically effective amount of at least one or two other active agent(s) selected from the group consisting of: Selective Serotonin Reuptake Inhibitors (SSRI), Fluvoxamine, Sulforaphane, Apigenin, Indole-3-cabinol, Baicalin, Bufalin, Quercetin, Curcumin, Nutrigenomic NRF2 Activators, inhibitors of NF-kappaB, and Prostacyclin Analogues, and Applied Therapeutics Aldose Reductase inhibitor AT-001.wherein the Corona Virus is COVID-19, a mutation of COVID-19, another Corona Virus with similar mechanisms of action to COVID-19, or ARDS. Any of the foregoing combinations of TTM and DEC, TTM and Zileuton, with other active agents, for administration by a variety of forms via different routes.

The foregoing description and accompanying figures illustrate the principles, preferred embodiments, and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments may be made by those skilled in the art without departing from the scope of the invention as defined by the following claims.

### CITATIONS

(1) Kang Y,ChenTet al. Cardiovascular manifestations and treatment considerations in Covid-19.Heart, April 2020.
(2) Guzik T et al. Covid-19 and the cardiovascular system. Cardiovasc. Res. April 2020.
(3) Li X et al.Clinical characterization of 25 death cases with COVID-19;A retrospective review of medicalrecords in a single center, Wuhan,China,Int J Infect Dis. May 2020.
(4) Zhao M et al. Advances in the relationship between corona virus infection and cardiovascular diseases. Biomed Pharmacother, May 2020.
(5) Tan W, Aboulhosen J.The cardiovascular burden of corona virus disease 2019 (COVID-19) with a focus on congenital heart disease.Int.J. Cardiol,June 2020.
(6) Munster VJ et al. Respiratory disease in rhesus macaques inoculated wit SARS-CoV-2.Nature, May 2020.
(7) Ligo M.et al. Single cell landscape of bronchoalveolar immune cells in patients with Covid-19.Nature Medicine, May 2020.
(8) Delgado-Roche L. Oxidative Stress as key player in SARS-CoV infection. Arch Med Res. April 2020.
(9) Sansome C., et al. Antioxidants as potential vectors controlling viral diseases, Antioxidants, May 2020.
(10) Hu Z, Yu F, Gong P, Qiu Y, Zhou W, Cui Y, Li J, Chen H. Subneurotoxic copper (II)-induced NF-κB-dependent microglial activation is associated with mitochondrial ROS. Toxicol Appl Pharmacol. 2014 Apr. 15;276(2):95-103.
(11) Khan G, Merajver S.Copper chelation in cancer therapy using tetrathiomolybdate: an evolving paradigm. Expert Opin Investig Drugs. 2009 April;18(4):541-8.
(12) McElwee MK, Song MO, Freedman JH. Copper activation of NF-kappaB signaling in HepG2 cells. J Mol Biol. 2009 Nov. 13;393(5):1013-21.
(13) Yang P, Gu H, Zhao Z, Wang W, Cao B, Lai C, Yang X, Zhang L, Duan Y, Zhang S, Chen W, Zhen W, Cai M, Penninger JM, Jiang C, Wang x. Angiotensin-converting enzyme 2 (ACE2) mediates influenza H7N9 virus-induced acute lung injury. Sci Rep. 2014 Nov. 13;4:7027.
(14) Geng Y-J. et al Pathophysiological characteristics and therapeutic approaches for Pulmonary Injury and Cardiovascular complications of Corona Virus Disease 2019 Cardiovasc. Pathol. Apr. 17, 2020.
(15) Ribeiro E.L et al. Diethylcarbamazine attenuates the development of carrageenan-induced lung injury in mice. Mediators of Inflammation, 2014
(16) Ackermann M et al. Pulmonary vascular endothelialitis, thrombosis and angiogenesis in COVID-19, NEJM, May 2020.
(17) Tan L. et al.Validation of predictors of disease severity and outcomes in COVID-19 patients: a descriptive and retrospective study. Med May 10, 2020.
(18) The Global Phosphorylation Landscape of SARS-CoV-2 Infection Mehdi Bouhaddou, Danish Memon, Bjoern Meyer, Kris M. White, Veronica V. Rezelj, Miguel C. Marrero, Benjamin J. Polacco, James E. Melnyk, Svenja Ulferts, Robyn M. Kaake, Jyoti Batra, Alicia L. Richards, Erica Stevenson, David E. Gordon, Ajda Rojc, Kirsten Obernier, Jacqueline M. Fabius, Margaret Soucheray, Lisa Miorin, Elena Moreno, Cassandra Koh, Quang Dinh Tran, Alexandra Hardy, Rémy Robinot, Thomas Vallet, Benjamin E. Nilsson-Payant, Claudia Hernandez-Armenta, Alistair Dunham, Sebastian Weigang, Julian Knerr, Maya Modak, Diego Quintero, Yuan Zhou, Aurelien Dugourd, Alberto Valdeolivas, Trupti Patil, Qiongyu Li, Ruth Hüttenhain, Merve Cakir, Monita Muralidharan, Minkyu Kim, Gwendolyn Jang, Beril Tutuncuoglu, Joseph Hiatt, Jeffrey Z. Guo, Jiewei Xu, Sophia Bouhaddou, Christopher J.P. Mathy, Anna Gaulton, Emma J. Manners, Eloy Félix, Ying Shi, Marissa Goff, Jean K. Lim, Timothy McBride, Michael C. O'Neal, Yiming Cai, Jason C.J. Chang, David J. Broadhurst, Saker Klippsten, Emmie De wit, Andrew R. Leach, Tanja Kortemme, Brian Shoichet, Melanie Ott, Julio Saez-Rodriguez, Benjamin R. tenOever, Dyche Mullins, Elizabeth R. Fischer, Georg Kochs, Robert Grosse, Adolfo García-Sastre, Marco Vignuzzi, Jeffery R. Johnson, Kevan M. Shokat, Danielle L. Swaney, Pedro Beltrao, Nevan J. Krogan PII: S0092-8674(20)30811-4 DOI: https://doi.org/10.1016/j.cell.2020.06.034 Reference: CELL 11493
(19) Hoffmann BR, Wagner JR, Prisco AR, Janiak A, Greene AS. Vascular endothelial growth factor-A signaling in bone marrow-derived endothelial progenitor cells exposed to hypoxic stress.Physiol Genomics. 2013 Nov. 1;45(21):1021-34.

What is claimed is:

1. A method of treating a Corona Virus or Influenza in a patient in need thereof, comprising administering to the patient a therapeutically effective amount of a copper chelator comprising a tetrathiomolybdate (TTM) salt and at least one co-drug, wherein the Corona Virus is COVID-19, a mutation of COVID 19, another Corona Virus, or variant(s) with similar mechanisms of action to Covid 19 or Acute Respiratory Distress Syndrome (ARDS).

2. The method of claim 1, wherein the copper chelator comprises a TTM salt according to:
X(MoS₄),
wherein:
X is (2Li)^{30 2}, (2K)⁺², (2Na)⁺², Mg⁺², Ca⁺², or {[N⁺(R¹)(R²)(R³)(R⁴)][N⁺(R⁵)(R⁶)(R⁷)(R⁸)]}, R¹, R², R³, R⁵, R⁶, and R⁷ are independently H, or optionally substituted group selected from the group consisting of alkyl, alkenyl, alkynyl, aryl, heteroaryl, cycloalkyl, heterocycloalkyl, aralkyl, alkylaralkyl, heteroaralkyl, cycloalkyl alkyl, and heterocycloalkyl alkyl; and
R⁴ and R⁸ are absent or independently H, or optionally substituted group selected from the group consisting of alkyl, alkenyl, alkynyl, aryl, heteroaryl, cycloalkyl, heterocycloalkyl, aralkyl, alkylaralkyl, heteroaralkyl, cycloalkyl alkyl, and heterocycloalkyl alkyl.

3. The method of claim 2, wherein the copper chelator comprises [NH₄]₂MoS₄.

4. The method of claim 1, wherein the copper chelator comprising a TTM salt and the at least one co-drug are administered orally.

5. The method of claim 4, wherein the copper chelator comprising a TTM salt is in an oral form administered orally in a delayed release preparation that releases the copper chelator comprising a tetrathiomolybdate salt after the oral form passes the stomach of the patient.

6. The method of claim 1, wherein the copper chelator comprising a TTM salt is administered intravenously and the at least one co-drug is administered orally, intravenously, or by inhalation.

7. A method of treating a Corona Virus or Influenza in a patient in need thereof, comprising administering to the patient a therapeutically effective amount of a copper chelator comprising a tetrathiomolybdate (TTM) salt and a therapeutically effective amount of at least one other active agent selected from the group consisting of: Selective Serotonin Reuptake Inhibitors (SSRI), Diethylcarbamazine (DEC), Zileuton, Fluvoxamine, Sulforaphane, Apigenin, Indole-3-cabinol, Baicalin, Bufalin, Quercetin, Curcumin, Nutrigenomic NRF2 Activators, inhibitors of NF-kappaB, Prostacyclin Analogues, Invermectin, and Applied Therapeutics Aldose Reductase inhibitor AT-001.

8. The method of claim 7, wherein the Corona Virus is COVID-19 a mutation of COVID 19, another Corona Virus with similar mechanisms of action to Covid 19, or ARDS.

9. The method of claim 7, wherein the copper chelator comprises a TTM salt according to:
X(MoS₄),
wherein:
X is (2Li)⁺², (2K)⁺², (2Na)⁺², Mg⁺², Ca⁺², or {[N⁺(R¹)(R²)(R³)(R⁴)][N⁺(R⁵)(R⁶)(R⁷)(R⁸)]}, R¹, R², R³, R⁵, R⁶, and R⁷ are independently H, or optionally substituted group selected from the group consisting of alkyl, alkenyl, alkynyl, aryl, heteroaryl, cycloalkyl, heterocycloalkyl, aralkyl, alkylaralkyl, heteroaralkyl, cycloalkyl alkyl, and heterocycloalkyl alkyl; and
R⁴ and R⁸ are absent or independently H, or optionally substituted group selected from the group consisting of alkyl, alkenyl, alkynyl, aryl, heteroaryl, cycloalkyl, heterocycloalkyl, aralkyl, alkylaralkyl, heteroaralkyl, cycloalkyl alkyl, and heterocycloalkyl alkyl.

10. The method of claim 7, wherein the copper chelator comprises [NH₄]₂MoS₄.

11. The method of claim 7, wherein the copper chelator comprising a TTM salt and the at least one other active agent are administered separately.

12. The method of claim 7, wherein the copper chelator comprising a TTM salt is administered orally and the at least one other active agent is administered orally, intravenously, or by inhalation.

13. The method of claim 7, wherein the at least one other active agent is DEC, and the copper chelator comprising a TTM salt is administered with the DEC in a manner selected from the group consisting of:
(a) oral administration of a combination oral form of the copper chelator comprising a TTM salt and a 5-lipoxygenase inhibitor comprising DEC, both of which are within either an enteric coated capsule in which the copper chelator comprising a TTM salt is separated from the DEC by a coating or sealed in in a separate compartment of the capsule from the DEC, or a tablet in which the copper chelator comprising a TTM salt and the DEC are separated by a barrier or coating;
(b) oral administration of a first oral form being a combination oral form of the copper chelator comprising a TTM salt with DEC or Zileuton, the copper chelator comprising a TTM salt being separately sealed or otherwise isolated from DEC or Zileuton, the first oral form being a capsule without an enteric coating, and a second oral form of the required proton pump inhibitor, such proton pump inhibitor protecting the TTM salt from stomach acid;
(c) oral administration of three separate oral forms, optionally packaged together: a first oral form comprising the copper chelator comprising a TTM salt without an enteric coating, a second oral form comprising DEC, and a third oral form comprising a proton pump inhibitor;
(d) oral administration of a first oral form including the copper chelator comprising a TTM salt, without enteric coating, and a second oral from, which includes a combination of DEC and a proton pump inhibitor;
(e) oral administration of a first oral form including a combination of the copper chelator comprising a TTM salt and a proton pump inhibitor, each sealed from each other, no enteric coating, and a second oral form comprising DEC;
(f) a combination of administration routes, wherein the copper chelator comprising a TTM salt is administered in an oral form, either with or without an enteric coating, and DEC is administered in an inhaled or intravenous form;
(g) a combination of administration routes, wherein the copper chelator comprising a TTM salt is administered in an intravenous form and DEC is administered as an oral, inhaled or intravenous form;
(h) a combination of administration routes, wherein the copper chelator comprising a TTM salt is administered in an intravenous form and DEC is administered in an inhaled form comprising at least one of Beraprost, Fluvoxamine, or Sulforaphane; and
(i) a combination of administration routes, wherein the copper chelator comprising a TTM salt is administered in an intravenous form and DEC is administered in an inhaled form comprising Fluvoxamine or Sulforaphane through an inhaler.

14. The method of claim 13, wherein, the copper chelator comprising a TTM salt is administered with DEC as one or more oral forms and one or more additional active agent is administered in one or more oral forms or in combination with said one or more oral forms: Fluvoxamine, Sulforaphane, Selective Serotonin Reuptake Inhibitors (SSRI), Apigenin, Indole-3-carbinol (i3c), Baicalin, Bufalin, Quercetin, Curcumin, Applied Therapeutics Aldose Reductase inhibitor AT-001, Nutrigenomic NRF2 Activators, inhibitors of NF-kappaB, Invermectin, and Prostacyclin Analogues.

15. The method of claim 7, wherein the copper chelator comprising a TTM salt and DEC are administered with Ivermectin in a first oral firm comprising the DEC and Ivermectin and a second oral form comprising the copper chelator comprising a TTM salt with an enteric coating.

16. The method of claim 7, wherein the copper chelator comprising a TTM salt and DEC are administered with sulforaphane, Fluvoxamine, or a combination thereof in a first oral firm comprising the DEC, sulforaphane, Fluvoxamine or combination thereof, and a second oral form comprising the copper chelator comprising a TTM salt with an enteric coating.

17. The method according to claim 7, further comprising administering another antiviral composition, an antibody treatment, or another Corona Virus treatment, to the patient.

18. The method according to claim 7, further comprising administering one or more drug targeting a Corona virus through anti-viral mechanism, antiviral protein, corona virus antibody, or investigational nucleotide analog with broad-spectrum antiviral activity to the patient.

19. A method of treating a Corona Virus or Influenza in a patient in need thereof, comprising administering to the patient a therapeutically effective amount of Diethylcarbamazine (DEC) and a therapeutically effective amount of at least one other active agent selected from the group consisting of: Selective Serotonin Reuptake Inhibitors (SSRI), Fluvoxamine, Sulforaphane, Apigenin, Indole-3-cabinol, Baicalin, Bufalin, Quercetin, Curcumin, Nutrigenomic NRF2 Activators, inhibitors of NF-kappaB, Prostacyclin Analogues, and Applied Therapeutics Aldose Reductase inhibitor AT-001, wherein the Corona Virus is COVID-19, a mutation of COVID-19, another Corona Virus with similar mechanisms of action to COVID-19, or ARDS.
